(19) United States
(12) Patent Application Publication
(10) Pub. No.: US 2009/0221683 A1
(43) Pub. Date: Sep. 3, 2009

(54) Combination of CXCR4 Antagonist and Morphogen to Increase Angiogenesis

(75) Inventor: Douglas W. Losordo, Chicago, IL (US)

Correspondence Address: EDWARDS ANGELL PALMER & DODGE LLP, P.O. BOX 55874, BOSTON, MA, 02205, US

(73) Assignee: Caritas St.Elizabeth Medical Center of Boston, Inc, Boston, MA (US)

(21) Appl. No.: 12/090,046

(22) PCT Filed: Oct. 18, 2006
(86) PCT No.: PCT/US06/40957

§ 371 Date: Jun. 23, 2008

Related U.S. Application Data
(60) Provisional application No. 60/728,133, filed on Oct. 18, 2005.

(51) Int. Cl. A61K 31/7088

(52) U.S. Cl. 514/44 R

(57) ABSTRACT

The present invention generally provides methods for preventing, treating or reducing the severity of symptoms associated with tissue ischemia by administering a CXCR4 antagonist in combination with at least one nucleic acid encoding a morphogen or effective fragment thereof. In one embodiment, the methods include elevating peripheral blood endothelial progenitor cells (EPCs), bone marrow-derived stem cells (BMSCs), or both cell types. The invention has a wide spectrum of applications including reducing or eliminating tissue ischemia, especially tissue ischemia associated with a myocardial infarct (heart attack).

### CROSS-REFERENCE TO RELATED APPLICATION

The present application claims priority from U.S. Provisional Patent Application No. 60/728,133 entitled Combination of CXCR4 antagonist and morphogen to increase angiogenesis as filed on Oct. 18, 2005, the disclosure of which is incorporated by reference.

### FIELD OF THE INVENTION

The present invention relates to methods for preventing, treating or alleviating symptoms associated with tissue ischemia and related conditions. In one aspect, the invention provides methods for treating tissue ischemia by administrating to a mammal a therapeutically effective amount of at least one CXCR4 antagonist and at least one morphogen or an effective fragment thereof. A preferred morphogen is human sonic hedgehog (hSHh). The invention has a wide spectrum of useful applications including treating tissue ischemia, particularly tissue ischemia associated with a myocardial infarction (MI).

### BACKGROUND OF THE INVENTION

There is recognition that cardiovascular disease is a major cause of mortality in the United States and claims approximately 1 million lives each year. The majority of cardiovascular deaths are due to coronary artery disease (CAD). In addition to these dire mortality statistics, CAD results in new or recurrent myocardial ischemia (MI) in approximately 1.1 million individuals each year in the United States. Of these individuals approximately 40% will go on to develop significant heart failure. A variety of strategies has been used in attempts to address the clinical consequences of loss of cardiac muscle mass, including medical treatment, revascularization to limit residual ischemia and, more recently, cardiac resynchronization therapy. In spite of these measures there were nearly one million hospitalizations for congestive heart failure in 2000, underscoring the fact that few if any therapies can fully compensate for the loss of myocardial integrity.

The feasibility of using gene therapy to enhance angiogenesis has received recognition. For example, there have been reports that angiogenesis can facilitate treatment of ischemia in a rabbit model and in human clinical trials. Particular success has been achieved using VEGF-1 administered as a balloon gene delivery system. Successful transfer and sustained expression of the VEGF-1 gene in the vessel wall subsequently augmented neovascularization in the ischemic limb (Takeshita, et al., Laboratory Investigation, 75:487-502 (1996); Isner, et al., Lancet, 348:370 (1996)). In addition, it has been reported that direct intramuscular injection of DNA encoding VEGF-1 into ischemic tissue induces angiogenesis, providing the ischemic tissue with increased blood vessels (Tsurumi et al., Circulation, 94:3281-3290 (1996)).

Intramuscular transfection of genes encoding single angiogenic cytokines may constitute a new treatment strategy for such patients (Isner and Losordo 1999). This strategy is designed to promote the development of supplemental collateral blood vessels that will constitute endogenous bypass conduits around occluded native arteries to prevent cardiac ischemia, and potentially restore myocardial function. There have been reports that VEGF gene transfer may have a favorable effect on the recovery of myocardial ischemia in humans (Losordo, D. W. et al. Circulation, 105, 2012-8. (2002).) and there are indications that this may be associated with an increase in myocardial performance (Vale, P. R., Losordo, D. & Milliken, C. Circulation, 104, 11-361 (2001); Vale, P. R. et al. Circulation, 103: 2138-43. (2001).

There have been reports that certain morphogens can be used to prevent, treat or reduce the severity of certain myocardial indications and related conditions. (See, e.g., WO2005/028616 and U.S. Ser. No. 60/490,064, the disclosures of which are incorporated herein by reference.) As disclosed, preferred treatment methods include administering a therapeutically effective amount of a nucleic acid encoding at least one morphogen; or an effective fragment thereof. Preferred morphogens include the human Sonic Hedghog (Shh), human Desert Hedgehog (Dhh), and human Indian Hedgehog (Ihh) proteins. As disclosed, the method can be used alone or in combination with other active agents including EPCs.

Small molecule therapeutic agents have also been found to be useful in the treatment of ischemia. For example, CXCR4 antagonists, originally developed for the treatment of HIV have recently been found to be effective in the treatment of ischemia, including ischemia associated with acute myocardial infarction (AMI) (WO2005/002522).

The use of endothelial progenitor cells (EPCs) as therapeutic agents for ischemia, including cardiac ischemia, has been investigated. There have been reports that EPCs isolated from peripheral blood can be augmented in response to certain cytokines and/or tissue ischemia. EPCs have been shown to home to, and incorporate into sites of neovascularization (i.e., sites of new blood vessel formation). (Asahara T. et al (1997) Science 275:964-967; Asahara T. et al. (1999) Circ Res. 85:221-228; Takahashi T. et al. (1999) Nat. Med. 5:434-438; Asahara T et al. (1999) EMBO J. 18:3964-3972; Kalka C. et al. (2000) Circ Res. 86:1198-1202; Kalka C. et al. (2000) Ann. Thorac. Surg. 70:829-834; Shi Q. et al. (1998) Blood 92:362-367; Hatzopoulos A. K. et al. (1998) Development 125:1457-1468; Gunsilius E. et al. (2000) Lancet 355:1688-1691; Gehling U. M. et al. (2000) Blood 95:3106-3112; Crosby J. R. et al. (2000) Circ Res. 87:728-730; Moldovan N. I. et al. (2000) Circ Res. 87:378-384; Murohara T. et al. (2000) J. Clin. Invest. 105:1527-1536). Such cells have been shown to incorporate into sites of neovascularization in adult mammals. This observation is consistent with the notion of postnatal vasculogenesis. Some studies have also shown that functional recovery after myocardial infarction is enhanced if the formation of new vessels is augmented.

Recently, EPCs have been investigated as therapeutic agents. In these studies of “supply-side angiogenesis,” EPCs harvested from the peripheral circulation have been expanded ex vivo and then administered to animals with limb (Kalka C. et al. (2000) Proc. Natl. Acad. Sci. USA 97:3422-3427) or myocardial (Kawamoto A. et al. (2001) Circulation 103: 634-637) ischemia to successfully enhance neovascularization. Physiological evidence of neovascular function in these preclinical animal models includes a high rate of limb salvage and improvement in myocardial function.

Other progenitor cell populations have been reported. For instance, D. Losordo and co-workers have isolated bone marrow-derived stem cells (BMSCs) having low or undetectable levels of certain cell markers such as CD34, FLK-1, tie-1 and others sometimes associated with endothelial, neuronal and smooth muscle cells. (See, e.g., WO 2005/042723 and U.S. Ser. No. 60/515,320, the disclosures of which are incorporated herein by reference.) It has been proposed that the BMSCs can be used therapeutically to prevent, treat or reduce the severity of certain cardiovascular indications.

Alternative methods for promoting angiogenesis are desirable for a number of reasons. For example, it is believed that native endothelial progenitor cell (EPC) number and/or viability decreases over time. Thus, in certain patient populations, e.g., the elderly, EPCs capable of responding to angiogenic proteins may be limited. Also, such patients may not respond well to conventional therapeutic approaches.

There have been reports that at least some of these problems can be reduced by administering isolated EPCs to patients and especially those undergoing treatment for ischemic disease. However, this suggestion is believed to be prohibitively expensive as it can require isolation and maintenance of patient cells. Moreover, handling of patient cells can pose a significant health risk to both the patient and attending personnel in some circumstances.

Granulocyte macrophage colony stimulating factor (GM-CSF) has been shown to exert a regulatory effect on granulocyte-committed progenitor cells to increase circulating granulocyte levels (Gasson, J. C., Blood 77:1131 (1991). In particular, GM-CSF acts as a growth factor for granulocyte, monocyte and eosinophil progenitors.

Administration of GM-CSF to human and non-human primates results in increased numbers of circulating neutrophils, as well as eosinophils, monocytes and lymphocytes. Accordingly, GM-CSF is believed to be particularly useful in accelerating recovery from neutropenia in patients subjected to radiation or chemotherapy, or following bone marrow transplantation. In addition, although GM-CSF is less potent than other cytokines, e.g., FGF, in promoting EC proliferation, GM-CSF activates a fully migrating phenotype. (Bussolino, et al., J. Clin. Invent., 87:986 (1991).

It would be desirable to have methods that can be used to modulate vascularization (eg., increase angiogenesis) in a mammal and especially a human patient. It would be particularly desirable to have methods that modulate the mobilization of EPCs, BMSCs (or both) to increase angiogenesis.

### SUMMARY OF THE INVENTION

The present invention generally relates to methods for preventing, treating, or reducing the severity of symptoms associated with tissue ischemia, and formulations and kits for use in the methods of the instant invention. In one aspect, the invention provides methods for preventing, treating or alleviating symptoms associated with tissue ischemia that include administering to a mammal a therapeutically effective amount of at least one CXCR4 antagonist and at least one nucleic acid that encodes at least one morphogen (or effective fragment thereof). Methods of the invention can be used alone or in combination with other agents such as those that promote the proliferation of endothelial progenitor (EPC) cells. The invention has many important uses including treating tissue ischemia, especially ischemia associated with acute myocardial infarction (AMI).

We have found that it useful to combine bone marrow cell mobilization therapy with an angiogenic gene therapy as a way to help prevent, treat, or alleviate symptoms associated with tissue ischemia. More specifically, it has been found that the combined therapy can mobilize progentior cells (particularly EPCs, bone marrow-derived stem cells (BMSCs), or in some cases both cell types) by administering to a subject in need of such treatment at least one CXCR4 antagonist and at least one nucleic acid encoding at least one morphogen or effective fragment thereof. By combining the therapies, it is possible to substantially enhance presence of circulating progentitor cells in the subject mammal. Without wishing to be bound to theory, it is believed that by combining the bone marrow cell mobilization and gene therapies disclosed herein, rather than using each alone, it is possible to achieve cooperative effects, and in some cases additive or synergistic effects, that positively act to promote enhanced frequency of EPCs, BMSCs, or in some instances both cell types in the subject. Such an effect is further believed to enhance angiogenesis in patients in need of such treatment. It is further believed that angiogenic cytokine production is substantially improved in ischemic tissues subjected to methods of the invention. Practice of the invention can be used before, during or after incidence of tissue ischemia as needed.

In one embodiment, the present methods can be used during or after ischemia, particularly AMI, to enhance recruitment and incorporation of EPCs into sites where new vasculature is needed. Examples of such sites include those associated with damage to the myocardium and related tissue including supporting vasculature. In this embodiment, preferred practice of the invention reduces or eliminates many symptoms associated with AMI including, but not limited to, myocardial fibrosis and reduced left ventricle (LV) dilation. It is thus an object of the invention to provide effective treatments methods for the prevention, treatment, and/or alleviation of ischemia and/or ischemic injury, that include administering at least one CXCR4 antagonist and at least one nucleic acid encoding at least one morphogen. The method preferably promotes EPC mobilization and enhances neovascularization at and/or near sites of ischemia. In embodiments in which AMI is to be treated, such sites will often include peri-infarct myocardium.

More generally, the present invention provides methods for preventing, treating, and/or alleviating tissue ischemia that includes administering to a subject at least one optionally substituted cyclic polyamine as provided herein (see also e.g., U.S. Pat. No. 5,583,131) in combination with a nucleic acid that encodes a desired morphogen or effective fragment thereof (See, e.g., WO2005/028616). The invention also includes the use of the compounds employed in the methods of the invention for the manufacture of a medicament for use in the prevention, alleviation, and/or treatment of ischemia wherein the CXCR4 inhibitor is formulated for systemic, preferably subcutaneous administration, and the nucleic acid coding a morphogen or an active fragment of a morphogen is formulated for injection into the ischemic tissue, preferably intramuscular injection, more preferably for pericardial or intracardiac injection. The invention further includes a kit comprising the compounds employed in the methods of the invention for use in the prevention, alleviation, and/or treatment of ischemia wherein the CXCR4 inhibitor is formulated for systemic, preferably subcutaneous administration, and the nucleic acid coding a morphogen or an active fragment of a morphogen is formulated for injection into the ischemic tissue, preferably intramuscular injection, more preferably for pericardial or intracardiac injection.

A preferred cyclic polyamine is known to or can be shown to antagonize activity of the CXCR4 receptor. More particular cyclic polyamine antagonists feature about four or less cyclic polyamine groups, generally less than about three of such groups being preferred for most embodiments. More preferred CXCR4 antagonists are optionally substituted bicyclic polyamines in which each polyamine group is spaced from the other by a specific linker. A preferred linker includes an optionally substituted aryl group comprising about six carbon atoms. Particular CXCR4 antagonists are described below.

Acceptable CXCR4 antagonists for use with the invention preferably induce EPC mobilization according to one or more assays as provided herein. However in accord with the invention, it will be desirable to combine such use with administration of a nucleic acid encoding at least one morphogen or an effective fragment thereof. Such antagonists also desirably assist neovascularization in the subject, particularly at or near sites impacted by tissue ischemia. By the term “induction” is meant enhancing EPC mobilization and preferably also facilitating formation of new blood vessels in ischemic tissue when compared to a suitable control (ie. without administration of the antagonist). By “EPC mobilization” is meant a significant increase in the number of peripheral blood EPCs as determined by assays disclosed herein.

More particular methods according to the invention include administering to the mammal (a) a therapeutically effective amount of a CXCR4 antagonist and (b) a nucleic acid encoding at least one morphogen or effective fragment thereof, under conditions sufficient to elevate peripheral blood EPC counts when compared to a control in which the antagonist and nucleic acid administration has not been employed. Preferably, the amount of administered CXCR4 antagonist and nucleic acid encoding the morphogen is also capable of increasing neovascularization in the ischemic tissue when compared to the control. Even more preferably, the therapeutically effective amount of the CXCR4 antagonist and administered nucleic acid encoding the morphogen is sufficient to help maintain a detectable level of ischemic tissue function in the mammal and in some cases essentially preserving that function at or near normal levels as determined by the assays disclosed herein. A variety of methods for detecting and quantifying the blood EPC count, neovascularization and ischemic tissue function are known and include those assays discussed below and in the examples which follow. The method may further comprise identification of an individual suffering from, and/or prone to ischemia, especially cardiac ischemia, prior to subjecting the individual to the methods of the invention. The method can also include monitoring of the individual for indication(s) that ischemia has been prevented, alleviated, and/or treated.

In a particular embodiment of the method, the enhancement in EPC mobilization and typically also the increase in peripheral blood EPC count is at least about 20% and preferably from between 50% to 500% as determined by what is referred to herein as a “standard cultured EPC assay”. That assay generally detects and quantifies EPC number and is described in detail below.

In another particular embodiment, the amount of administered CXCR4 antagonist and nucleic acid encoding the morphogen or effective fragment thereof is sufficient to preserve the function of at least part of the myocardium in the subject, usually a mammal such as a primate, that has suffered or is suffering from AMI. Methods for detecting and quantifying heart function include conventional methods known in the field such as echocardiographic and hemodynamic measurements, particularly ventricle dilation (systolic and/or diastolic), fractional shortening, or both. Other suitable tests include conventional hemodynamic measurements of systolic pressure, end-diastolic pressure, +dP/dt, and −dP/dt.

Additionally suitable amounts of administered CXCR4 antagonist and nucleic acid encoding the morphogen or effective fragment include amounts sufficient to increase recruitment of EPCs to and/or increase capillary density in ischemic tissue by at least about 20% and preferably from between 50% to 500% as determined by what is referred to herein as a “standard hind limb ischemia assay” as discussed below.

As will become more apparent from the discussion and Examples which follow, the invention is flexible and can be used as the sole therapy or in combination with other methods, agents or therapies for modulating EPC proliferation. In one embodiment, the invention includes methods for treating tissue ischemia in a mammal in which the therapeutically effective amount of the CXCR4 antagonist and nucleic acid is co-administered with at least one other agent that promotes EPC proliferation. It is believed that in some embodiments, co-administration of the CXCR4 antagonist, the nucleic acid encoding the morphogen or effective fragment thereof and the third agent can further increase neovascularization in the ischemic tissue of the mammal by providing additive or synergistic effects when compared to administration of the CXCR4 antagonist and the nucleic acid alone. A preferred agent for promoting EPC proliferation includes those recognized endothelial cell mitogens. Specific agents are discussed below.

The invention further includes the use of the agents of the invention for the preparation of a medicament for the prevention, alleviation, and/or treatment of ischemia, wherein the medicament includes a CXCR4 antagonist; and a nucleic acid encoding a morphogen or an active fragment thereof, and at least one additional agent to promote EPC proliferation. The medicament can be formulated to allow the components of the medicament to be administered alone, or in combination with one or more of the other components. The invention also includes a kit including the agents of the invention for the preparation of a medicament for the prevention, alleviation, and/or treatment of ischemia, wherein the medicament includes a CXCR4 antagonist; and a nucleic acid encoding a morphogen or an active fragment thereof, and at least one additional agent to promote EPC proliferation.

Thus in one embodiment, the invention includes administering a therapeutically effective amount of at least one of the forgoing agents to further promote EPC and/or bone marrow stem cell (BMSC) proliferation in the subject. The agent that promotes the cell proliferation can be administered to the subject before, after or during administration of the CXCR4 antagonist and the nucleic acid encoding the morphogen or effective fragment thereof. The order in which the CXCR4 antagonist and the nucleic acid is provided to the subject is not important so long as intended results are achieved. Accordingly, and in one embodiment, the CXCR4 antagonist is provided prior to administration of the morphogen or fragment encoded by the nucleic acid. In another embodiment, the nucleic acid encoding the morphogen or effective fragment thereof is provided to the subject prior to administration of the CXCR4 antagonist. In yet another embodiment, co-administration of the CXCR4 antagonist and the nucleic acid encoding the morphogen or effective fragment thereof can be used to practice the invention. More specific administration approaches are discussed below.

In another invention embodiment, the methods provided herein can be used in conjunction with one or more other methods for preventing or treating tissue ischemia. Such methods include, but are not limited to, surgical procedures such as angioplasty, and conventional anti-clotting therapies such use of one or more of an anti-thrombolytic or anti-coagulants known in the field.

In another aspect, the invention provides a pharmaceutical product that is preferably formulated to modulate and especially to induce EPC mobilization in a subject. In one embodiment, the product includes a therapeutically effective amount of a CXCR4 antagonist and a nucleic acid encoding at least one morphogen or an effective fragment thereof that is preferably suitable for administration to a mammal and particularly a human patient in need of such treatment. In a specific embodiment, the product is provided as a sterile formulation and can optionally include a therapeutically effective amount of an agent that promotes EPC proliferation. Alternatively, the CXCR4 antagonist, the nucleic acid, and, optionally the agent that promotes EPC proliferation can be provided as physically separate pharmaceutical formulations. If desired, the pharmaceutical formulation can be provided in a kit or related format.

Further provided are methods for inducing new blood vessel growth in myocardial tissue of a mammal in need of such treatment comprising administering a therapeutically effective amount of at least one CXCR4 antagonist and a nucleic acid encoding at least one morphogen; or an effective fragment thereof.

Additionally provided is a method for increasing production of EPCs, BMSCs, or both cell types in a subject. In one embodiment, the method includes administering a therapeutically effective amount of at least one of a CXCR4 antagonist and a nucleic acid encoding at least one of an N-terminal portion of human hedgehog (Shh) protein, human desert hedgehog (Dhh) protein or human Indian Hedgehog (Ihh) protein. A preferred method further includes increasing presence of the EPCs, the BMSCs or both in the subject. The invention also includes the use of a CXCR4 antagonist; and a nucleic acid encoding a morphogen or an active fragment of a morphogen, for the preparation of a medicament to increase the presence of EPCs, BMSCs, or both in an individual preferably wherein the individual is in need of such a medicament. The medicament may further include other components. The medicament may be prepared for administration of the components separately or together. The invention further includes the components required to increase the production of EPCs, BMSCs, or both in the form of a kit.

Other aspects of the invention are disclosed infra.

### BRIEF DESCRIPTION OF THE DRAWINGS

FIG. 1 depicts a graph of the change in mononuclear cell (MNC) and endothelial progenitor cell (EPC) numbers after a single injection of AMD-3100.

FIGS. 2A-2B depict the results of echocardiographic measurements in mice injected with AMD-3100 after AMI. FIG. 2A depicts the measurement of LVDd (diastolic left ventricle dilation) and LVDs (systolic left ventricle dilation). FIG. 2B depicts the measurement of heart rate and fractional shortening.

FIGS. 3A-3C depict the results of hemodynamic measurements using a Millar catheter in mice injected with AMD-3100 after AMI. FIG. 3A depicts the measurement of LVSP (left ventricle systolic pressure) and LVEDP (left ventricle end-diastolic pressure). FIG. 3B depicts +dP/db. FIG. 3C depicts −dP/dt.

FIGS. 4A-4B depict the survival rate of mice injected with AMD-3100 after AMI.

FIGS. 5A-5D depict the results of immunohistochemistry in the border zone one and two weeks after AMI, with or without treatment with AMD-3100. The data are representative findings using an antibody to isolectin B4. FIG. 5A: AMD-3100, 1 week; FIG. 5B: AMD-3100, 2 weeks; FIG. 5C: control, 1 week; FIG. 5D: control, 2 weeks.

FIGS. 6A-6C depict the quantitation of capillary density at the border zone one and two weeks after injection with or without AMD-3100 after AMI. FIG. 6A: one week; FIG. 6B: two weeks FIG. 6C depicts data for one, two, and four weeks.

FIGS. 7A-7C depict the decrease in left ventricle fibrosis area two weeks after AMI, with or without treatment with AMD-3100. FIG. 7A: macroscopic view, AMD-3100; FIG. 7B: macroscopic view, control; FIG. 7C: quantitation of % fibrosis area.

FIG. 8 depicts the number of EPCs in peripheral blood one and two weeks after injection with AMD-3100 after AMI.

FIGS. 9A-9D depict the augmentation of recruitment of bone marrow derived EPCs 1 and 2 weeks after AMI, with or without treatment with AMD-3100. FIG. 9A: AMD-3100, 1 week; FIG. 9B: AMD-3100, 2 weeks; FIG. 9C: control, 1 week; FIG. 9D: control, 2 weeks.

FIGS. 10A-10D depict representative findings using counterstaining 1 and 2 weeks after AMI, with or without treatment with AMD-3100, in Tie2-LacZ/BMT mice. FIG. 9A: AMD-3100, 1 week; FIG. 9B: AMD-3100, 2 weeks; FIG. 9C: control, 1 week; FIG. 9D: control, 2 weeks.

FIGS. 11A-11B depict the quantitation of X-gal positive cells (i.e., bone-marrow derived endothelial cells) present in ischemic tissue after injection with AMD-3100 after AMI. FIG. 11A: one week; FIG. 11B: two weeks.

FIGS. 12A-12F depict representative results of fluorescence microscopy of ischemic myocardium 1 week after AMI, with or without treatment with AMD-3100. Cells were stained for BS lectin 1 and/or β-galactosidase (β-gal) to identify bone-marrow derived endothelial cells. FIG. 12A: AMD-3100, BS lectin 1; FIG. 12B: AMD-3100, β-gal; FIG. 12C: AMD-3100, BS lectin 1 and β-gal double stain; FIG. 13D: control, BS lectin 1; FIG. 12E: control, β-gal; FIG. 13F: control, BS lectin 1 and β-gal double stain.

FIGS. 13A-13F depict representative results of fluorescence microscopy of intact myocardium 1 week after AMI, with or without treatment with AMD-3100. Cells were stained for BS lectin 1 and/or β-galactosidase (β-gal) to identify bone marrow derived endothelial cells. FIG. 13A: AMD-3100, BS lectin 1; FIG. 13B: AMD-3100, β-gal; FIG. 13C: AMD-3100, BS lectin 1 and β-gal double stain; FIG. 13D: control, BS lectin 1; FIG. 13E: control, β-gal; FIG. 13F: control, BS lectin 1 and β-gal double stain.

FIGS. 14A-14B depict representative results from 4-quadrant analysis of double positive cells between Sca-1 (x axis) and Flk-1 (y axis) for AMD-3100 treated and untreated EPCs isolated from peripheral blood, showing an increase in circulating EPCs 1 week after MI. FIG. 14A: using FACS analysis; FIG. 14B: using statistical analysis.

FIGS. 15A-15E depict the following: FIG. 15A: Representative 4-quadrant analysis of double positive cells between Sca-1 (x-axis) and Flk-1 (y axis) for AMD-3100-treated and untreated EPCs isolated from total BM cells using FACS analysis. FIG. 15B: Statistical analysis shows that AMD-3100 prevents decreasing EPCs in BM 1 week after MI. FIG. 15C: AMD-3100 upregulates expression of MMP-9 mRNA in BM mononuclear cells. Panel shows RT-PCR products for MMP-9, MMP-2 and GAPDH. FIG. 15D: Representative 4-quadrant analysis of double positive cells between Sca-1 (x axis) and Flk-1 (y axis) for AMD-3100-treated and untreated EPCs isolated from total BM cells in MMP-9^{-/-} mice 1 week after MI by using FACS analysis. FIG. 15E: Statistical analysis also shows no differences in BM EPCs 1 week after MI.

### DETAILED DESCRIPTION OF THE INVENTION

As discussed, the present invention provides methods for preventing, treating, or alleviating symptoms associated with tissue ischemia in a subject such as a human patient. In one embodiment, the methods include administering to the patient a therapeutically effective amount of at least one suitable CXCR4 antagonist in combination with at least one nucleic acid encoding at least one morphogen or effective fragment thereof. It will be appreciated that reference herein to combination therapy means that the CXCR4 antagonist and nucleic acid can be administered to the subject at the same time or at different times, by the same or different routes optionally with different numbers of administrations, to achieve intended treatment results. The invention has a wide spectrum of uses including preventing or reducing the severity of tissue damage after ischemia, especially AMI.

The present invention is flexible and can be adapted as needed to suit a desired therapeutic outcome. For example, and in one embodiment, the CXCR4 antagonist and the nucleic acid encoding the morphogen can be administered alone as the sole therapeutic agent or in combination with at least one other agent, preferably one that promotes EPC and/or BMSC proliferation.

As discussed, it is an object of the invention to provide methods for the mobilization of EPCs, BMSCs, or both and to assist neovascularization. A variety of methods for making, using, and identifying BMSCs have been disclosed (see, e.g., WO2005/042723) and are well known to those skilled in the art. BMSCs are highly plastic and can be induced to form a variety of cell types. Without wishing to be bound to theory, it is believed that EPC mobilization can be assisted by blocking activity of the CXCR4 receptor, especially such receptors expressed in or near bone marrow tissue. Mobilization of EPCs according to the invention helps to elevate peripheral blood EPC count, thereby making more EPCs available for formation of new blood vessels. Frequency of BMSCs is similarly increased. The increased availability of circulating EPCs, BMSCs or both cell types provided by the invention is believed to promote neovascularization in a wide variety of ischemic tissues in need of treatment including, but not limited to, the myocardium. Methods of the invention generally include administering to the patient a therapeutically effective amount of at least one CXCR4 antagonist e.g., preferably less than about five of same such as one, two or three of such antagonists, in combination with a nucleic acid that encodes the morphogen or active fragment thereof.

Thus in one invention embodiment, the method provided herein further includes the step of mobilizing in the subject at least one of the following: EPCs and BMSCs. The method can further include the step of arteriogenesis in the subject by at least about 10% as determined by capillary density assay. In addition, the method can further include the step of improving cardiac function by at least about 10% as measured by at least one of the following assays: LV diastolic, systolic dimensions, percent fractional shortening, echocardiography, hemodynamic measurement and LV dp/dt. Such methods of measurement are discussed in further detail in the Examples.

By the phrase “ischemic tissue” is meant damaged tissue having a deficiency in blood or blood vessels typically as the result of myocardial infarction, cerebrovascular ischemia, renal ischemia, pulmonary ischemia, limb ischemia, ischemic cardiomyopathy, ischemic organ (e.g., a transplanted organ) and myocardial ischemia. An individual in need of prevention, alleviation, and/or treatment of ischemia is prone to, suspected of having, or known to have tissue ischemic conditions such as those listed above. For example, individuals with circulatory problems due to diabetes or other conditions that damage circulation may be prone to or suspected of having ischemic tissue even if no such tissue has been observed directly. Tissues after organ transplant may also be prone to ischemia. Individuals with cardiovascular disease can be prone to both ischemia and myocardial infarction.

A variety of methods for making, using, and identifying the BMSCs have been disclosed (see, e.g., WO 2005/042723). Such cells are highly plastic and can be induced to form a variety of cell types.

Administration of one or more CXCR4 antagonists according to the invention can be as needed and may be implemented before, during, or after detection of ischemic tissue in the subject. In many instances, treatment can begin if the subject is suspected to possess ischemic tissue such as after a heart attack. When administered in conjunction with an agent that promotes EPC proliferation, the CXCR4 antagonist may be administered at the same time, before, or preferably, after the agent that promotes EPC proliferation.

As discussed, methods of this invention have a wide spectrum of uses especially in the prevention or treatment of tissue damage resulting from tissue ischemia. Such tissue damage can arise as a direct result or indirectly arise from the ischemia. A particular type of tissue ischemia of interest is associated with one or more of acute myocardial infarction (referred to interchangeably herein as “myocardial infarction”, “MI”, “AMI”, or its common name, “heart attack”), trauma, graft rejection, cerebrovascular ischemia, renal ischemia, pulmonary ischemia, ischemia related to infection, limb ischemia, ischemic cardiomyopathy, cerebrovascular ischemia, and myocardial ischemia.

It will be appreciated however that the invention is not limited to addressing these specific indications. That is, the methods provided herein are intended to enhance neovascularization of nearly any tissue, organ or physiological system in a subject mammal including those tissues and organs associated with circulatory system or the central nervous system, e.g., a limb, graft (e.g., muscle or nerve graft), or organ (e.g., heart, brain, kidney and lung). For instance, the ischemia may adversely impact heart or brain tissue as often occurs in cardiovascular disease or stroke, respectively, in which case the invention can be used to prevent or treat the ischemia afflicting those tissues.

The CXCR4 antagonist can be administered to the subject before, during or after detection of tissue ischemia in the subject. Similarly the nucleic acid encoding a morphogen or active fragment thereof can be administered at any time relative to the detection of tissue ischemia. However, the invention is not tied to demonstration of such ischemia in the subject. That is, the invention methods can be employed, for instance, when the subject has experienced a myocardial infarct. Thus in one embodiment, the CXCR4 antagonist can be administered not more than about a month after detection of the ischemia (or after the infarct) for example, 1, 2, 3, 4, 5, 6, 7, 8, 9, 10, 11, 12, 14, 16, 18, 20, 22, or 24 hours thereafter. However in other embodiments, more robust treatment methods may be indicated and require administering the CXCR4 antagonist to the subject during or not long after (e.g., less than a few days later including several hours after) detection of the ischemia or infarct occurrence.

The methods of this invention are suitable for tissue ischemia in a variety of animals including mammals. The term “mammal” is used herein to refer to a warm-blooded animal such as a rodent, rabbit, or a primate and especially a human patient. Specific rodents and primates of interest include those animals representing accepted models of human disease including the pig, chimp, sheep, goat, horse, mouse, rat, rabbit, and monkey. Particular human patients of interest include those who have, are suspected of having, or are at risk of having ischemic tissue. Methods to identify such individuals is well known to those of ordinary skill in the art and are discussed herein.

Conditions that facilitate or assist formation of ischemic tissue are known in the field and include, but are not limited to, a surgical manipulation (e.g., restenosis associated with angioplasty) or a medical condition such as AMI. Ischemic tissue is often associated with an ischemic vascular disease such as those specific conditions and diseases discussed below.

As discussed above, it has been found that particular bicyclic polyamines can be used to promote neovascularization of ischemic tissue. Without wishing to be bound by theory it is believed that preferred administration of such compounds reduces or block CXCR4 receptor activity especially in bone marrow. Reduction or in some instances a cessation in receptor activity augments EPC mobilization from the bone marrow into the peripheral blood. In accordance with the present invention, such bicyclic polyamines are believed to function as potent CXCR4 antagonists which can be used to enhance recovery of ischemic tissue.

The term “CXCR4”, as used herein, shall be understood to refer to the CXCR4 chemokine receptor, a receptor in the GPCR (G-protein coupled receptor) gene family, which is expressed by cells in the bone marrow, immune system and the central nervous system. In response to binding its ligand SDF-1 (stromal cell-derived factor-1), CXCR4 is thought to trigger the migration and recruitment of immune cells, as well as the homing of stem cells (e.g., EPCs). The receptor is believed to enhance downstream signaling by several different pathways. As a GPCR, CXCR4 binding of SDF-1 activates G-protein mediated signaling, including downstream pathways such as ras, and PI3 kinase. PI3 kinase activated by SDF-1 and CXCR4 plays a role in lymphocyte chemotaxis in response to these signals. One endpoint of CXCR4 signaling is the activation of transcription factors such as AP-1 and chemokine regulated genes. JAK/STAT signaling pathways also appear to play a role in SDF-1/CXCR4 signaling.

The structure of the human CXCRF4 receptor is known. See GenBank Accession Nos. NM_{—}003467 and NP_{—}003458, respectively. The nucleotide and polypeptide sequences of human SDF-1 are set forth in GenBank Accession Nos. NM_{—}000609 and NP_{—}000600, respectively. See Hwang, J. H. et al. (2003) J. Clin. Endocrinol. Metab. 88(1):408-416; Babcock, G. J. et al. (2003) J. Biol. Chem. 278(5):3378-3385; Barbouche, R. et al. (2003) J. Biol. Chem. 278(5):3131-3136; Adams, G. B. et al. (2003) Blood 101(1):45-51; Lapham, C. K. et al. (2002) J. Leukcoc. Biol. 72(6):1206-1214; Zhou, Y. et al. (2002) J. Biol. Chem. 277(51):49481-49487; Sun, Y. et al. (2002) J. Biol. Chem. 277(51):49212-49219; Bachelder, R. E. et al. (2002) Cancer Res. 62(24),:7203-7206; Barbero, S. et al. (2002) Ann. N.Y. Acad. Sci. 973:60-69; Rey, M. et al. (2002) J. Immunol. 169(10):5410-5414; Basmaciogullari, S. et al. (2002) J. Virol. 76(21):10791-10800; Konig, R. R. et al. (2002) J. Virol. 76(21):10627-10636; Martinez-Caceres, E. M. et al. (2002) Mult. Scler. 8(5):390-395; Odemis, V. et al. (2002) J. Biol. Chem. 277(42):39801-39808; Moriuchi, M. et al. (2002) J. Infect. Dis. 186(8):1194-1197; Kollet, O. et al. (2002) Blood 100(8):2778-2786; Libura, J. et al. (2002) Blood 100(7):2597-2606; Honczarenko, M. et al. (2002) Blood 100(7):2321-2329; Ptasznik, A. et al. (2002) J. Exp. Med. 196(5):667-678; Estes, J. D. et al. (2002) J. Immunol. 169(5):2313-2322; Farzan, M. et al. (2002) J. Biol. Chem. 277 (33):29484-29489; Fotopoulos, G. et al. (2002) Proc. Natl. Acad. Sci. U.S.A. 99(14):9410-9414; Peled, A. et al. (2002) Stem Cells 20(3):259-266; Habasque, C. (2002) Mol. Hum. Reprod. 8(5):419-425; Zhou, N. et al. (2002) J. Biol. Chem. 277(20):17476-17485; Lu, M. et al. (2002) Proc. Natl. Acad. Sci. U.S.A. 99(10):7090-7095; Valenzuela-Femandez, A. et al. (2002) J. Biol. Chem. 277(18):15677-15689; Schrader, A. J. et al. (2002) Br. J. Cancer 86(8):1250-1256; Nguyen, D. H. and Taub, D. (2002) J. Immunol. 168(8):4121-4126; Salvucci, O. et al. (2002) Blood 99(8):2703-2711; Juffermans, N. P. et al. (2002) J. Infect. Dis. 185(7):986-989; Taichman, R. S. et al. (2002) Cancer Res. 62(6):1832-1837; Zamarchi, R. et al. (2002) Clin. Exp. Immunol. 127(2):321-330; Arthos, J. et al. (2002) Virology 292(1):98-106; Ferraro, G. A. et al. (2001) AIDS Res. Hum. Retroviruses 17(13):1241-1247; Ullrich, C. K. et al. (2000) Blood 96(4):1438-1442; Poznansky, M. C. et al. (2000) Nat. Med. 6(5):543-548; Secchiero, P. et al. (2000) J. Immunol. 164(8):4018-4024; Cheng, Z. J. et al. (2000) J. Biol. Chem. 275(4):2479-2485; Lalani, A. S. et al. (1999) Science 286(5446):1968-1971; Sotsios, Y. et al. (1999) J. Immunol. 163(11):5954-5963; Gupta, S. K. and Pillariseffi, K. (1999) J. Immunol. 163(5):2368-2372; Klein, R. S. et al. (1999) J. Immunol. 163(3):1636-1646; Ling, K. et al. (1999) Proc. Natl. Acad. Sci. U.S.A. 96(14):7922-7927; Yasukawa, M. et al. (1999) J. Immunol. 162(9):5417-5422; Zou, Y. R. et al. (1998) Nature 393(6685):595-599; Tachibana, K. et al. (1998) Nature 393(6685):591-594; Caruz, A. et al. (1998) FEBS Lett. 426(2):271-278; Moriuchi, M. et al. (1997) J. Immunol. 159(9):4322-4329; Bleul, C. C. et al. (1996) Nature 382(6594):829-833; Choe, H. et al. (1996) Cell 85(7):1135-1148; Lu, Z. H. et al. (1995) J. Biol. Chem. 270(44):26239-26245; Loetscher, M. et al. (1994) J. Biol. Chem. 269(1):232-237; Nomura, H. et al. (1993) Int. Immunol. 5(10):1239-1249; Jazin, E. E. et al. (1993) Regul. Pept. 47(3):247-258; Herzog, H. et al. (1993) DNA Cell Biol. 12(6):465-471; Federsppiel, B. et al. (2993) Genomics 16(3):707-712 (1993); Bleul, C. C. et al. (1996) Nature 382(6594):829-33); Cheng, Z. et al. (2000) J. Biol. Chem. 275(4):2476-2485; Dutt, P. et al. (1998) J. Immunology. 161: 3652-3658; Wang, J. F. et al. (2000) Blood 95(8):2505-13; Ling K. et al. (1999) Cell Biology 96:7922-7927; Vicente-Manzanares, M. et al. (1999) Immunology 163:4001-12; Ganju, R. K. et al. (1998) Biological Chemistry 273(36):23169-175; and Zhang, X. F. et al. (2001) Blood 97(11):3342-3348; and references cited therein.

The structure and function of the SDF-1 ligand is also known (also referred to herein as SDF-1α). See Glodek, A. M. et al. (2003) J. Exp. Med. 197(4):461-473; Roland, J. et al. (2003) Blood 101(2):399-406; Adamns, G. B. (2003) Blood 101(1):45-51; Sun, Y. et al. (2002) J. Biol. Chem. 277(51):49212-49219; Krug, A. et al. (2002) J. Immunol. 169(11):6079-6083; Barbero, S. et al. (2002) Ann. N.Y. Acad. Sci. 973:60-69; Nance, C. L. and Shearer, W. T. (2002) Clin. Immunol. 105(2):208-214; Libura, J. et al. (2002) Blood 100(7):2597-2606; Honczarenko, M. (2002) Blood 100(7):2321-2329; Netelenbos, T. (2002) J. Leukoc. Biol. 72(2):353-362; Farzan, M. et al. (2002) J. Biol. Chem. 277(33):29484-29489; Okabe, S. et al. (2002) Exp. Hematol. 30(7):761-766; Langford, D. (2002) J Neuroimmunol. 127(1-2):115-126; Inngjerdingen, M. et al. (2002) Blood 99(12):4318-4325; Lee, Y. (2002) Blood 99(12):4307-4317; Peled, A. (2002) Stem Cells 20(3):259-266; Wright, N. et al. (2002) J. Immunol. 168(10):5268-5277; Valenzuela-Femandez, A. (2002) J. Biol. Chem. 277(18):15677-15689; Schrader, A. J. et al. (2002) Br. J. Cancer 86(8):1250-1256; Salvucci, O. et al. (2002) Blood 99(8):2703-2711; Taichman, R. S. et al. (2002) Cancer Res. 62(6):1832-1837; Zaitseva, M. et al. (2002) J. Immunol. 168(6):2609-2617; Casamayor-Palleja, M. et al. (2002) Blood 99(6):1913-1921; Phillips, R. and Ager, A. (2002) Eur. J. Immunol. 32(3):837-847; Tresoldi, E. et al. (2002) J. Infect. Dis. 185(5):696-700; Riabov, G. S. et al. (2002) Genetika 38(2):278-280; Lataillade, J. J. et al. (2002) Blood 99(4):1117-1129; Nanki, T. and Lipsky, P. E. (2001) Cell. Immunol. 214(2):145-154; Lathey, J. L. et al. (2001) J. Infect. Dis. 184(11):1402-1411; Bajetto, A. et al. (2001) J. Neurochem. 77(5):1226-1236; Poznansky, M. C. et al. (2000) Nat. Med. 6(5):543-548; Ghezzi, S. et al. (2000) Biochem. Biophys. Res. Commun. 270(3):992-996; Cheng, Z. J. et al. (2000) J. Biol. Chem. 275(4):2479-2485; Luttichau, H. R. et al. (2000) J. Exp. Med. 191(1):171-180; Lalani, A. S. et al. (1999) Science 286(5446):1968-1971; Sotsios, Y. et al. (1999) J. Immunol. 163(11):5954-5963; Vicente-Manzanares, M. et al. (1999) J. Immunol. 163(7):4001-4012; Kozak, S. L. et al. (1999) J. Biol. Chem. 274(33):23499-23507; Su, S. B. et al. (1999) J. Immunol. 162(12):7128-7132; Weber, K. S. et al. (1999) Mol. Biol. Cell 10(4):861-873; Zaitseva, M. B. et al. (1998) J. Immunol. 161(6):3103-3113; Rubbert, A. et al. (1998) J. Immunol. 160(8):3933-3941; Bleul, C. C. et al. (1996) Nature 382(6594):829-833; and Shirozu, M. et al. (1995) Genomics 28(3):495-500; and references cited therein.

The term “CXCR4 antagonist”, as used herein, refers to a cyclic polyamine as represented by the following general formula set forth as formula (I) below.

Formula I

Z-linker-Z′ (1)

wherein Z is a cyclic polyamine containing 9-32 ring members of which 3-8 are nitrogen atoms;

said nitrogen atoms separated from each other by at least 2 carbon atoms,

wherein said heterocycle may optionally contain additional heteroatoms besides nitrogen and/or may be fused to an additional ring system.

Z′ may be embodied in a form as defined by Z above, or alternatively may be of the formula

—N(R)—(CR₂)ₙ—X

wherein

each R is independently H or straight, branched or cyclic alkyl (1-6C),

n is 1 or 2, and

X is an aromatic ring, including heteroaromatic rings, or is a mercaptan;

A “linker” represents a bond, alkylene (1-6C) or may comprise aryl, fused aryl, oxygen atoms contained in an alkylene chain, or may contain keto groups or nitrogen or sulfur atoms.

In general, preferred embodiments of Z and Z′ according to Formula I are cyclic polyamine moieties having from about 9 to about 24 carbon atoms and include from about 3 to about 5 nitrogen atoms.

Preferred cyclic polyamines in accord with the invention include the following: 1,5,9,13-tetraazacyclohexadecane; 1,5,8,11,14-pentaazacyclohexadecane; 1,4,8,11-tetraazacylotetradecane; 1,5,9-triazacylcododecane; 1,4,7,10-tetraazacyclododecane; and the like, including such cyclic polyamines which are fused to an additional aromatic or heteroaromatic rings and/or containing a heteroatom other than nitrogen incorporated in the ring. Embodiments wherein the cyclic polyamine contains a fused additional cyclic system or one or more additional heteroatoms are described in U.S. Pat. No. 5,698,546 incorporated hereinabove by reference.

More preferred cyclic polyamines according to the invention are bicyclic molecules in which each cyclic group is spaced from the other by a convenient linker Examples Include the Following Molecules:
3,7,11,17-tetraazabicyclo(13.3.1)heptadeca-1(17),13,15-triene;
4,7,10,17-tetraazabicyclo(13.3.1)heptadeca-1(17),13,15-triene;
1,4,7,10-tetraazacyclotetradecane; 1,4,7-triazacyclotetradecane; and
4,7,10-triazabicyclo(13.3.1)heptadeca-1(17),13,15-triene.

See U.S. Pat. Nos. 5,021,409; 5,583,131; 5,698,546; 5,817,807; application Ser. No. 09/111,895 filed Jul. 8, 1998; 5,612,478; 5,756,728; 5,801,281; 6,365,583, and 5,606,053 (disclosing useful related compounds), the disclosures of which are incorporated herein by reference.

Preferred linker moieties for spacing the cyclic polyamine group include those wherein the linker is a bond, or wherein the linker includes an aromatic (aryl) moiety. Preferably, the linker is includes two C₂₋₆ alkylene groups, preferably methylene moieties, that flank the aromatic group. Preferred linking groups include the methylene bracketed forms of 1,3-phenylene, 2,6-pyridine, 3,5-pyridine, 2,5-thiophene, 4,4′-(2,2′-bipyrimidine); 2,9-(1,10-phenanthroline) and the like. A particularly preferred linker is 1,4-phenylene-bis-(methylene).

An especially preferred bicyclic polyamine for use with the present invention includes a 2,2′-bicyclam; 6,6′-bicyclam group. See U.S. Pat. No. 5,583,131, and in particular 1,1′-[1,4-phenylene-bis(methylene)]-bis-1,4,8,11-tetraazacyclotetradecane, as set forth in U.S. Pat. No. 5,021,409, and designated herein AMD-3100. AMI-3100, a reversible, competitive inhibitor of SDF-loc binding to CXCR4, is a white to off-white powder with a molecular weight of 502.79 Da and the empirical formula C₂₈H₅₄N₈. Its chemical structure is shown below.

The structure of AMD-3100 is shown below as Formula II.

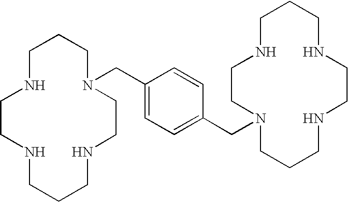

Additional cyclic polyamines for use according to the invention include the following specific compounds:
N-[1,4,8,11-tetraazacyclotetradecanyl-1,4-phenylenebis(methylene)]-2-aminomethyl)pyridine;
7,7′-[1,4-phenylenebis(methylene)]bis-4,7,10,17-tetraazabicyclo-[13.3.1]heptadeca-1(17),13,15-triene;
7,7′-[1,4-phenylenebis(methylene)]bis-3,7,11,17-tetraazabicyclo[13.3.1]heptadeca-1(17),13,15-triene;
1,1′-[1,3-phenylenebis(methylene)]-bis-1,4,8,11-tetra-azacyclotetradecane;
1,1′-[1,4-phenylenebis(methylene)]-bis-1,4,8,11-tetra-azacyclotetradecane;
1,1′-[1,4-phenylene-bis-(methylene)]-bis-1,4,7,10-tetraazacyclotetradecane;
1,1′-[1,3-phenylene-bis-(methylene)]-bis-1,4,7,10-tetraazacyclotetradecane;
11,11′-(1,2-propanediyl)bis-1,4,8,11-tetraazacyclotetradecane;
N-[4-(1,4,7-triazacyclotetra-decane)-1,4-phenylenebis(methylene)]-2-(aminomethyl)pyridine;
N-[7-(4,7,10-triazabicyclo[13.3.1]heptadeca-1(17),13,15-triene)-1,4-phenylenebis(methylene)]-2-(aminomethyl)pyridine;
N-[7-(4,7,10,17-tetraazabicyclo[13.3.1]heptadeca-1(17),13,15-triene)-1,4-phenylenebis(methylene)]-2-(aminomethyl)pyridine; and
N-[4-[4,7,10,17-tetraazabicyclo[13.3.1]heptadeca-1(17),13,15-triene]-1,4-phenylenebis(methylene)]-2-(aminomethyl)pyridine.

The present invention is compatible with one or a combination of different administration routes. For instance, and in one embodiment, compounds of the invention may be prepared in the form of prodrugs, i.e., protected forms which release the compounds of the invention after administration to the subject. Typically, the protecting groups are hydrolyzed in body fluids such as in the bloodstream thus releasing the active compound or are oxidized or reduced in vivo to release the active compound. A discussion of prodrugs is found in Smith and Williams Introduction to the Principles of Drug Design, Smith, H. J.; Wright, 2.sup.nd ed., London (1988).

Preferred cyclic polyamines for use with the invention can be readily identified by those in the field, typically by testing compounds in a what is referred to herein as a CXCR4 activity assay. Preferred assay formats are cell-based assay. Thus a cell-based CXCR4 activity assay (referred to herein as a “standard receptor binding assay”) typically includes providing a cell that expresses CXCR4, contacting the cell with stromal cell derived factor-1 (SDF-1) (or a receptor binding fragment thereof) along with the test compound. The test compound is typically assayed for its ability to antagonize SDF-1 mediated CXCR4 signaling. A test compound can also be assayed for the ability to antagonize SDF-1 binding to CXCR4 in vitro. Cyclic polyamines identified as potential CXCR4 antagonists using such assays can then be tested, for example, using methods described herein, particularly in the Examples, for the ability to mobilize EPCs into the peripheral blood circulation, for the ability to induce recruitment of bone marrow derived EPCs to sites of tissue ischemia, and/or for the ability to preserve function of ischemic tissue.

More preferred receptor antagonists of the invention reduce or completely block binding of SDF-1 (or a receptor binding fragment thereof) to the CXCR4 receptor, typically by at least about 25%, more preferably at least about 35%, most preferably at least about 50% when compared to full-length SDF-1. Methods for detecting and quantifying such activity are known and include the foregoing standard receptor-binding assay.

The above-described compounds can be prepared for use with the invention as pharmaceutically acceptable equivalents such as acid addition salts, solvates, hydrates, and metal complexes thereof. Suitable acid addition salts include salts of inorganic acids that are biocompatible, including HCl, HBr, sulfuric, phosphoric and the like, as well as organic acids such as acetic, propionic, butyric and the like, as well as acids containing more than one carboxyl group, such as oxalic, glutaric, adipic and the like. Typically, at physiological pH, the compounds of the invention will be in the forms of the acid addition salts. Particularly preferred are the hydrobromides. In addition, when prepared as purified forms, the compounds may also be crystallized as the hydrates.

If desired, one or a combination of the foregoing cyclic polyamines can be used in combination with one or more other antagonists of the CXCR4 receptor. For example, see ALX40-4C, T22, and T140 (See also Scozzafava, A. et al. (2002) J Enzyme Inhib. Med. Chem., 17(2):69-76; Fujii, N. et al. (2003) Expert Opin. Investig. Drugs 12(2):185-95; Agrawal, L. et al. (2001) Expert Opin. Ther. Targets 5(3):303-326; Pohlmann, S, and Doms, R. W. (2002) Curr. Drug Targets Infect. Disord. 2(1):9-16; De Clercq, E. (2002) Med. Res. Rev. 22(6):531-65; Starr-Spires, L. D. and Collman, R. G. (2002) Clin. Lab. Med. 22(3):681-701).

As discussed, it is an object of the invention to administer one or more of the forgoing CXCR4 antagonists with at least one nucleic acid (eg., less than five, preferably one, two, or three nucleic acids) that encodes the morphogen or effective fragment thereof. A wide variety of suitable morphogens and effective fragments thereof have been disclosed in WO 2005/028616.

In broad terms, morphogens are recognized amino acid sequences whose concentration is read by cells as positional information to a pre-determined landmark or beacon in certain cells. Such sequences help control pattern formation in a large field of adjacent tissue. See generally Alberts, B. et al. (1989) in the Molecular Biology of the Cell, 2^{nd} Ed. Garland Publishing, New York, N.Y. (discussing morphogen function). As used herein, “morphogen” or related phrase such as “morphogenic protein” means one of the following mammalian: Sonic hedgehog (SHh), Desert hedgehog (DHh) and Indian hedgehog (IHh); or an effective fragment thereof. Preferred morphogens are endogenous to a primate and especially a human. A more preferred morphogen is human SHh. It is well within the ability of those skilled in the art to identify effective fragments of morphogenic proteins.

There are reports that most morphogens and especially SHh are protein signaling molecules important for vertebrate patterning. In particular, SHh has been shown to be involved in the morphogenesis of many vertebrate organ systems. The sequence of many mammalian morphogens has been disclosed. For example, the human SHh sequence (nucleic acid and protein) has been reported eg., by Margio, V. (1995) eg., accession number L38518 (version L38518.1; GI: 663156) which reports a 1576 basepair human SHh nucleic acid sequence and corresponding encoded protein. It will be appreciated that any nucleic acid or protein sequence information not specifically disclosed herein can be readily accessed at Genbank, EMBL and/or SWISS-PROT. Using this information, a DNA or RNA segment encoding the desired may be chemically synthesized or, alternatively, such a DNA or RNA segment may be obtained using routine procedures in the art, e.g, PCR amplification. See also Pola, R. et al. (2003) Circulation 108: 479-485 in which an especially preferred SHh vector construct is disclosed.

As disclosed by the WO 2005/028616 application, by the phrase “effective fragment” is meant a nucleic acid or amino acid sequence that has at least about 70%, more preferably at least about 80%, even more preferably at least about 90% and most preferably at least about 95% of that function. By an “effective fragment” of a morphogen or related phrase is meant a portion of a morphogen protein or nucleic acid encoding same that has at least about 70%, more preferably at least about 80%, most preferably at least about 90% of the activity of the corresponding full-length protein or nucleic acid as determined by what is referred to herein as a “standard heart LV function assay”. Extensive structure-function analyses have been preformed on the hedgehog genes and proteins for use in the instant invention. These provide substantial guidance regarding fragments that may or may not be useful in the instant invention. A preferred version of that assay includes performing at least one of and preferably all of the following steps:
(a) directly injecting into a rodent subjected to LAD ligation a solution that includes therein a nucleic acid (about 1 μg/ml to about 5 mg/ml) encoding the desired portion of the morphogen,
(b) allowing the rodent to recover from the LAD ligation for at least about few hours, preferably about a few days or more, more preferably about a few weeks up to about one to two months; and
(c) evaluating function of the rodent heart e.g., by performing transthoracic echocardiography or related procedure.

Also contemplated for use with the present invention are morphogen “derivatives” which include a morphogen analog, including a modified active fragment of a morphogen analog, having substantial identity to the corresponding full-length or active fragment of a morphogen. Preferred derivatives are at least about 90% identical to the full-length morphogen as determined eg., by inspection or with the aid of a suitable computer program such as BLAST, FASTA or related programs, preferably at least about 95% identical. Suitable analogues include protein sequences having one or more conservative amino acid substitutions with respect to the corresponding full-length morphogen (eg., allelic variants). By “conservative” amino acid substitution is replacement of one amino acid residue for another having similar chemical properties (eg., replacing tyrosine with phenylalanine). The concept of conservative substitutions is well understood by those of ordinary skill in the art.

Methods for administering the nucleic acid to a mammal and particularly direct injection to or near the heart and associated tissue have been disclosed. See e.g., U.S. Pat. Nos. 5,830,879; 6,258,787; 6,121,246; RE37,933, 5,851,521 and 5,106,386; the disclosures of which are incorporated herein by reference. At least a portion of such methods are expected to be useful for direct injection into non-cardiac or peri-cardiac tissue.

In one embodiment, the method includes administering along with one or more of the CXCR4 antagonists a therapeutically effective amount of a nucleic acid encoding an N-terminal portion of at least one of human hedgehog (SHh) protein, human desert hedgehog (DHh) protein and human Indian Hedgehog (IHh) protein.

As also disclosed by WO2005/028616 application, there are reports that Human SHh is synthesized as a 45 kDa precursor protein that is cleaved autocatalytically to yield two “effective fragments” ie., (1) a 20 kDa N-terminal fragment that is responsible for all known hedgehog signaling activity; and (2) a 25 kDa C-terminal fragment that contains the autoprocessing activity. The N-terminal fragment is reported to consist of amino acid residues 24-197 of the full-length precursor sequence. The N-terminal fragment is thought to remain membrane-associated through the addition of a cholesterol at its C-terminus. The addition of the cholesterol is catalyzed by the C-terminal domain during the processing step. See eg., U.S. Pat. Nos. 6,132,728; 6,281,332; 6,444,793; and 6,288,048 as well as WO 95/18856; and WO 96/17924.

The complete amino acid sequences of the human DHh and human IHh proteins have been reported by Genbank as accession nos. BC033507 and BC034757, respectively. Also disclosed are the corresponding cDNA sequences. Significant relationship between the N-terminal fragments of the human SHh, human DHh, and human IHh proteins has been reported. (See, e.g., U.S. Pat. No. 6,444,793).

By the term “N-terminal portion” of the human SHh protein is meant between from about 400 bp to about 700 bp amino terminal domain coding sequence of the human SHh gene, preferably about the 600 bp amino terminal domain coding sequence. By the phrase “N-terminal portion” of the human Dhh and human Ihh proteins is meant less than about an 800 bp amino terminal domain coding sequence, preferably less than about 700 bp, more preferably between from about 300 bp to about 600 bp of the amino terminal domain coding sequence for the human Dhh and human Ihh proteins. Fragment lengths are generally measured from the N-terminus of the mature protein (before autocatalysis). Various other effective N-terminal fragments that encompass the N-terminal moiety are considered within the presently claimed invention. Publications disclosing these sequences, as well as certain of their chemical and physical properties, include WO 95/23223; WO 95/18856; and WO 96/17924; and U.S. Pat. No. 6,444,793. Preferred of such encoded N-terminal portions of the human SHh, DHh, and IHh proteins are those that exhibit at least 70%, preferably 80% of the activity of the corresponding full-length protein as determined by the standard heart LV function assay described above. For more information, see, e.g., WO 2005/028616.

Further effective portions of the human Shh, Dhh, and Ihh proteins are contemplated and include those are at least 80% identical to the N-terminal portion of the proteins specified above, preferably at least about 90% identical or more such as 95% identical over the length of the portion. Specifically included are allelic variants of such proteins as well as conservative amino substitutions of such proteins (eg., alanine for serine, glycine for alanine, etc.)

See the following references for disclosure relating to still further morphogens and effective fragments thereof suitable for use with the invention. Lee et al. (1994) Science 266:1528-1537; Porter et al. (1995) Nature 374:363-366); Bumcrot, D. A., et al. (1995) Mol. Cell. Biol. 15:2294-2303; Ekker, S. C. et al. (1995) Curr. Biol. 5:944-955; and Lai, C. J. et al. (1995) Development 121:2349-2360).

Table I of the WO 2005/028616 application provides the amino acid sequence of an especially preferred N-terminal amino portion of the human Shh gene sequence. Illustrative N-terminal portions of the human Dhh (B) and human Ihh gene (C) sequences are also shown.

As described elsewhere herein, a CXCR4 antagonist can be administered in conjunction with the nucleic acid encoding the morphogen or effective fragment thereof, and optionally with an additional agent that promotes the proliferation of EPCs. The use of such proliferation-promoting agents can provide a larger pool of EPCs that can be mobilized from the bone marrow. Agents that promote the proliferation of EPCs can be of any type, including small molecules, peptides, peptidomimetics, nucleic acids, or other types of compounds or agents.

In one embodiment, an agent that promotes the proliferation of EPCs (also referred to herein as an “EPC proliferation promoting agent”, or simply a “proliferation promoting agent”), includes the protein granulocyte macrophage colony stimulating factor (GM-CSF), granulocyte colony stimulating factor (G-CSF), GRO-β (a CXC cytokine), Flt-3 ligands, glutathione S-transferase inhibitors, angiotensin or analogs thereof, CD26 inhibitors, VLA4 inhibitors, kit ligands, vascular endothelial growth factor (VEGF), Steel factor (SLF, also known as Stem cell factor (SCF)), hepatocyte growth factor (HGF, scatter factor), Angiopoietin-1, Angiopoietin-2, macrophage colony stimulating factor (M-CSF), colony stimulating factor (CSF), acidic fibroblast growth factor (aFGF), and basic fibroblast growth factor (bFGF).

In embodiments in which the methods of the invention employ use of GM-CSF, it will be appreciated that the natural or recombinantly prepared protein having substantial identity to an amino acid sequence of human GM-CSF will be referred to.

See published international application WO 86/00639, which is incorporated herein by reference. Recombinant human GM-CSF is hereinafter also referred to as “hGM-CSF”.

See also Kim, C. H. and Broxmeyer, H. E. (1998) Blood, 91: 100; Turner, M. L. and Sweetenham, J. W., Br. J. Haematol. (1996) 94: 592; Aiuuti, A. et al. (1997) J. Exp. Med. 185: 111; Bleul, C. et al. (1996) J. Exp. Med. 184: 1101; Sudo, Y. et al. (1997) Blood, 89: 3166; as well as references disclosed therein, disclosing additionally suitable EPC proliferation promoting agents.

In another embodiment of the invention, a CXCR4 antagonist and the nucleic acid encoding the morphogen or effective fragment thereof may be administered in conjunction with a therapeutically effective amount of an inhibitor of vascular endothelial growth factor-mediated vascular permeability, for example, an inhibitor of a src-family kinase.

The natural or recombinantly prepared proteins, and their functional equivalents used in the method of the invention are preferably purified and substantially cell-free, which may be accomplished by known procedures.

Additional protein and nucleic sequences relating to the factors disclosed herein including SDF-1 can be obtained through the National Center for Biotechnology Information (NCBI)— Genetic Sequence Data Bank (Genbank). In particular, sequence listings can be obtained from Genbank. See generally Benson, D. A. et al. (1997) Nucl. Acids. Res. 25:1 for a description of Genbank. Protein and nucleic sequences not specifically referenced can be found in Genbank or other sources disclosed herein.

In accord with the methods of this invention, a CXCR4 antagonist can be administered with a nucleic acid encoding morphogen or an effective fragment thereof to a mammal and particularly a human patient in need of such treatment. As an illustration, CXCR4 antagonist as well as therapeutic compositions including same are preferably administered parenterally. More specific examples of parenteral administration include subcutaneous, intravenous, intra-arterial, intramuscular, and intraperitoneal, with subcutaneous being preferred. In some embodiments, a CXCR4 antagonist is used to augment the effects of locally administered agents for neovascularization.

In embodiments of this invention in which parenteral administration is selected, the CXCR4 antagonist will generally be formulated in a unit dosage injectable form (solution, suspension, emulsion), preferably in a pharmaceutically acceptable carrier medium that is inherently non-toxic and preferably non-therapeutic. Examples of such vehicles include without limitation saline, Ringer's solution, dextrose solution, mannitol, and normal serum albumin. Neutral buffered saline or saline mixed with serum albumin are exemplary appropriate vehicles. Non-aqueous vehicles such as fixed oils and ethyl oleate may also be used. Additional additives include substances to enhance isotonicity and chemical stability, e.g., buffers, preservatives and surfactants, such as Polysorbate 80. The preparation of parenterally acceptable protein solutions of proper pH, isotonicity, stability, etc., is within the skill of the art.

Preferably, the product is formulated by known procedures as a lyophilizate using appropriate excipient solutions (e.g., sucrose) as a diluent.

Suitable dosage ranges for the compounds of formula (I) vary according to these considerations, but in general, the compounds are administered in the range of about 0.1 μg/kg-5 mg/kg of body weight; preferably the range is about 1 μg/kg-300 μg/kg of body weight; more preferably about 10 μg/kg-100 μg/kg of body weight. For a typical 70-kg human subject, thus, the dosage range is from about 0.7 μg-350 mg; preferably about 700 μg-21 mg; most preferably about 700 μg-7 mg. Frequency of dosing will have an effect on the amount of the compound administered. Frequency will depend on a number of factors including, but not limited to, complexity of dosing method, half-life of the compound, activity of the compound, and other factors well known to those skilled in the art. For example, a composition to be administered as a single dose may be administered in a higher quantity per dose than a compound to be administered multiple times. Also, dosages may be higher when the compounds are administered orally or transdermally as compared to, for example, intravenous administration.

Typical in vivo dosages for polypeptides such as SDF-1 will be guided by intended use however between from about 1 μg/kg/day to about 100 μg/kg/day will often be useful. Use of more specific dosages will be guided by parameters well known to those in this field such as the specific condition to be treated and the general health of the subject. See also U.S. Pat. No. 5,578,301 for additional methods of administering GM-CSF.

Reference herein to an “effective amount” and especially a “therapeutically effective amount” when used to describe an amount of nucleic acid encoding morphogen means a sufficient amount of nucleic acid delivered to the desired cells or tissue to produce an adequate level of the morphogen or effective fragment, i.e., levels capable of inducing endothelial cell growth and more generally, blood vessel growth. Thus, the important aspect is the level of morphogen expressed. Accordingly, one can use multiple transcripts or one can have the nucleic acid coding sequence under the control of a promoter that will result in high levels of expression. In an alternative embodiment, nucleic acid coding sequence would be under the control of a factor that results in extremely high levels of expression, e.g., tat and the corresponding tar element. For instance, administration of between about 1 pg/g organ weight to 1 mg/organ weight will suffice for many invention embodiments. Similar considerations are relevant to determining an “effective amount” or a “therapeutically effective amount” of a CXCR4 antagonist. It is noted that absolute amount of a compound required to produce the desired effect alone may be different, preferably higher, than that required to produce the desired effect than in combination with other agents.

Methods for making and using a variety of morphogen encoded by suitable expression vectors have been described in WO 2005/028616 and references disclosed therein including WO 95/23223, WO 95/18856, WO 96/17924; and U.S. Pat. No. 6,444,793.

It will be understood that the term “co-administration” is meant to describe preferred administration of at least two compounds or agents disclosed herein to the mammal, i.e., administration of one compound or agent in conjunction with, subsequent to, or prior to administration of the other compound or agent. It will be further understood that the term “in conjunction with” does not require administration of the compounds or agents in the same composition, by the same route of administration, or even at approximately the same time.

In embodiments in which co-administration of a DNA encoding a protein, e.g., an encoded EPC proliferation promoting protein, is desired, the nucleic acid encoding same can be administered to a blood vessel perfusing the ischemic tissue via a catheter, for example, a hydrogel catheter, as described by U.S. Pat. No. 5,652,225, the disclosure of which is herein incorporated by reference. The nucleic acid also can be delivered by injection directly into the ischemic tissue using the method described in WO 97/14307. If desired, such methods can also be used to administer nucleic acid encoding the morphogen or effective fragment thereof.

The nucleotide sequence of numerous EPC proliferation promoting proteins, are readily available through a number of computer databases, for example, GenBank, EMBL and Swiss-Prot. Using this information, a DNA segment encoding the desired may be chemically synthesized or, alternatively, such a DNA segment may be obtained using routine procedures in the art, e.g, PCR amplification.

The term “therapeutically effective amount” refers to a sufficient amount of a compound, e.g., a small molecule, protein or nucleic acid delivered to produce an adequate therapeutic response in a subject, e.g., levels capable of inducing EPC mobilization into the peripheral blood circulation, as determined by standard assays disclosed throughout this application.

As used herein, “treatment” of a subject includes the application or administration of a therapeutic agent to a subject, e.g., a subject suffering from tissue ischemia (e.g., from a mycardial infarction), with the purpose of curing, healing, alleviating, relieving, altering, remedying, ameliorating, improving, or affecting the ischemia and/or the damage to the tissue resulting from the ischemia. It will be understood by those of skill in the art that a “method of treatment” does not require a complete “cure”, or a complete absence of symptoms resulting from the ischemic event. A “method of treatment” refers to any method wherein the performance of the method leads to a beneficial effect in the subject on whom the method is performed.

By the term “standard EPC culture assay” or related term is meant an assay that includes at least one of and preferably all of the following steps.
a) isolating mononuclear cells from about 0.5 ml peripheral blood of the mammal,
b) culturing the cells in a suitable dish or plate in medium for several days and usually for about 4 days, at 37° C.,
c) staining the cells with acLDL-Dil and BS-1 lectin, and
d) quantitating double positive cells as being indicative of EPCs.

More specific disclosure relating to the standard EPC culture assay can be found in the discussion and Examples that follow.

Reference herein to a “standard hind limb ischemia assay” or related term is meant to denote a conventional assay for inducing hind limb ischemia in accepted animal models and particularly the mouse or rabbit. Disclosure relating to conducting the assay can be found in Couffinhal, T. et al. (1998) Am. J. Pathol., infra; and Takeshita, S. et al. (1994) J. Clinical. Invest. 93: 662.

In an illustrative treatment approach, a patient in need of treatment is selected. The patient is subsequently treated with between from about 0.1 micrograms/kg body weight to about 10,000 micrograms/kg body weight of a particular CXCR4 antagonist such as AMD-3100. The antagonist is administered by injection (eg., subcutaneous) more than once per week and preferably two, three or four times a week as indicated. Naked plasmid DNA encoding a desired morphogen such as a suitable N-terminal portion of the human Shh gene is administered by direct injection to an ischemic site in need of treatment (eg., intramyocardial injection to or near the site of an infarct). Typically, between from about 10 micrograms of plasmid/subject to about 10,000 micrograms plasmid/subject will be useful. Treatment efficacy can be monitored as needed such as within about a week following treatment or between from about a week to about several weeks (eg., about 4 weeks post treatment).

In embodiments in which treatment of a myocardial infarct is desired, treatment can be monitored by one or a combination of conventional approaches such as echocardiography, hemodynamic measurement, LV diastolic, systolic, percent fractional shortening and related approaches. A particular treatment approach will be effective if the result is at least about a 5% increase one or more of these indicators of heart function, preferably about a 10% increase relative to a suitable control (ie., a patient who has not received treatment according to the invention).

The present invention is further illustrated by the following examples. These examples are provided to aid in the understanding of the invention and are not construed as a limitation thereof. The contents of all references, patents and published patent applications cited throughout this application, as well as the figures, are incorporated herein in their entirety by this reference.

### EXAMPLE

### Example 1

### Cultured EPC Assay

Animals used in this study were handled in accordance with the guidelines of the Animal Care and Use Committee at St. Elizabeth's Medical Center of Boston. AMD3100 (125 μg in 100 μl of saline) was administered to eight-week old male FVB/N mice (Jackson Lab, Bar Harbor, Me.) aged 8 weeks by subcutaneous single injection before sacrifice. Control mice received 100 μl of saline according to same procedures by a similar procedure. Both groups of mice were sacrificed to harvest, and peripheral blood was harvested from subgroups of mice at 1, 3, 12 and 24 hours after the injection. At each time points, blood was obtained from the heart immediately before sacrifice and separated by Histopaque-1083 (Sigma) density gradient centrifugation with a Histopaque-1083 (Sigma). Light-density mononuclear cells were harvested and, washed twice with Dulbecco's phosphate-buffered saline supplemented with to which 5 mM EDTA had been added. Contaminated red blood cells were hemolyzed using ammonium chloride solution (Stem Cell Technologies).

Peripheral blood mononuclear cells (PBMNCs) were isolated from 500 μl of peripheral blood obtained from each mouse. PBMNCs were cultured in EBM media (Clonetics, San Diego, Calif.) supplemented with 5% fetal bovine serum (FBS), antibiotics, and growth factors (EPC medium) on four 4-well glass slides coated with rat plasma vitronectin (Sigma) in 0.5% gelatin solution. Four days after culture, EPCs, recognized as attaching spindle-shaped cells, were assayed by co-staining with acetylated LDL (acLDL)-DiI (Biomedical Technologies, Stoughton, Mass.) and Bandeiraea simplicifolia (BS-1) lectin I (Vector Laboratories, Burlingame, Calif.), characteristics indicators of endothelial lineage, and conjugated with FITC (Sigma). Fluorescent microscopy identified double-positive cells as EPCsEPCs were identified as double-positive cells under fluorescent microscopy. The numbers of double-positive cells per sample, measured in fifteen fields under 20× magnification, was were counted per sample in a blinded manner.

More specific details of the cultured EPC assay can be found in Takahashi, T. et al. (1999) Nat. Genet. 5:434-438, incorporated herein by reference. Such methods can be used for study of the compositions and methods of the instant invention.

### Example 2

### Change of MNC and EPC Numbers By 24 Hours After a Single Injection of AMD-3100

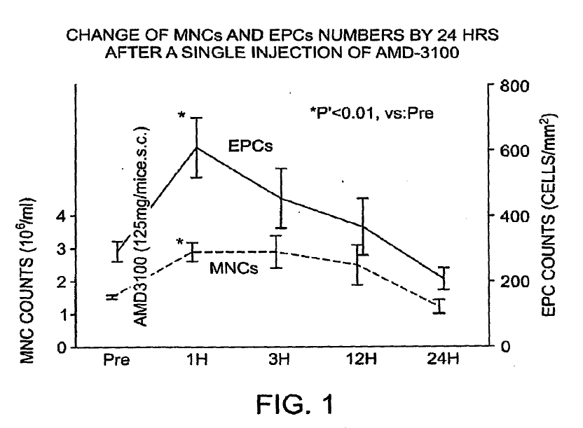

Mice were injected as described in Example 1. As shown in FIG. 1, the numbers of both MNCs and EPCs rapidly increased after AMD-3100 injection, resulting in maximal MNCs and EPCs 1 hour after drug administration (613±89 cells/mm² for AMD-3100 vs. 292±30 for control, P<0.01). The number of EPCs, identified by acLDL uptake and BS-1 lectin reactivity, increased at one hour after injection and returned to baseline within 24 hours after injection. These values returned to baseline within 24 hours (n=5 experiments). Such methods can be used for study of the compositions and methods of the instant invention. It is expected that use of the compositions and methods of the invention would also increase the number of MNCs and EPCs.

### Example 3

### Functional Study of AMD-3100 Injection After Myocardial Infarction

Eight-week old male FVB mice were anesthetized with sodium pentobarbital (50 mg/kg IP). The animals were intubated orally with a 22G IV catheter and artificially ventilated with a respirator (Harvard Apparatus). A left intercostal thoracotomy was performed and the ribs were retracted with 5-0 polypropylene sutures. After the pericardium was opened, the left anterior descending (LAD) branch of the left coronary artery was ligated proximal to the bifurcation between the LAD and the diagonal branch using 8-0 polypropylene sutures and visualized with a dissecting microscope. Positive end-expiratory pressure was applied to fully inflate the lungs, and the chest was closed with 7-0 polypropylene sutures. After the closure of chest wall, mice randomly received either a single subcutaneous injection of 125 μg AMD3100 (AMD group) or saline (control group). After physiological assessment at one, two and four weeks after MI, a subgroup of the mice were euthanized by intravenous administration of a lethal dose of pentobarbital.

Transthoracic echocardiography (SONOS 5500, Hewlett Packard) was performed just before MI and one, two and four weeks after MI. Left ventricular diastolic (LVDd) and systolic (LVDs) dimensions and fractional shortening were measured at the mid-papillary muscle level. Hemodynamic measurements were obtained at two and four weeks after MI using a 1.0-French high-fidelity pressure catheter (Millar Instruments, Houston, Tex.) via the left carotid artery. LV systolic pressure and maximal positive (+dP/dt) and negative (−dP/dt) pressure development were recorded after stabilization of LV function and heart rate. Such methods can be used for study of the compositions and methods of the instant invention.

### Example 4

### AMD-3100 Reduces Left Ventricle Dilation and Improves Left Ventricle Systolic Function Two Weeks After AMI

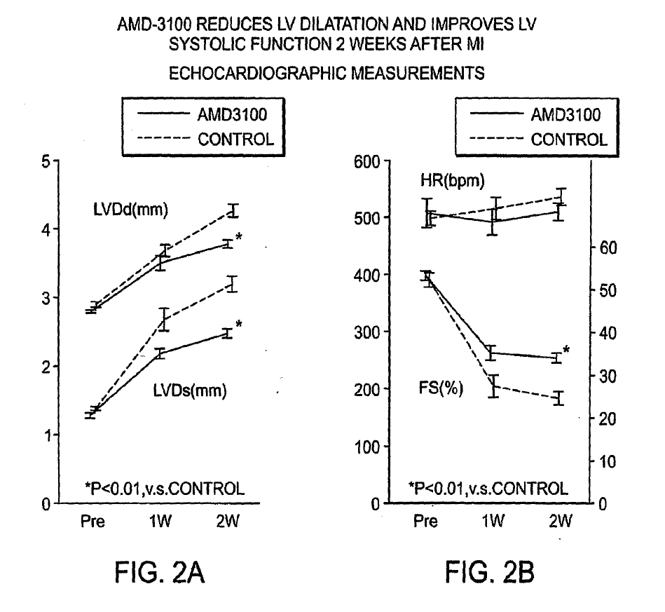

FIGS. 2A-2B shows the results of an echocardiographic study performed one and two weeks after AMI. Left ventricular systolic (2.5±0.1 mm for AMD-3100 vs. 3.2±0.1 mm for control) and diastolic (3.8±0.1 mm for AMD-3100 vs 4.3±0.1 mm for control) dimensions and fractional shortening (34±1% for AMD-3100 vs. 25±2% for control) in the AMD-3100 group were preserved two weeks after AMI, as compared to control (P<0.01). Such methods can be used for study of the compositions and methods of the instant invention. It is expected that use of the compositions and methods of the invention would also improve left ventricle dilation and improve left ventricle systolic function after AMI.

### Example 5

### AMD-3100 Preserves Left Ventricle Function Two Weeks After AMI

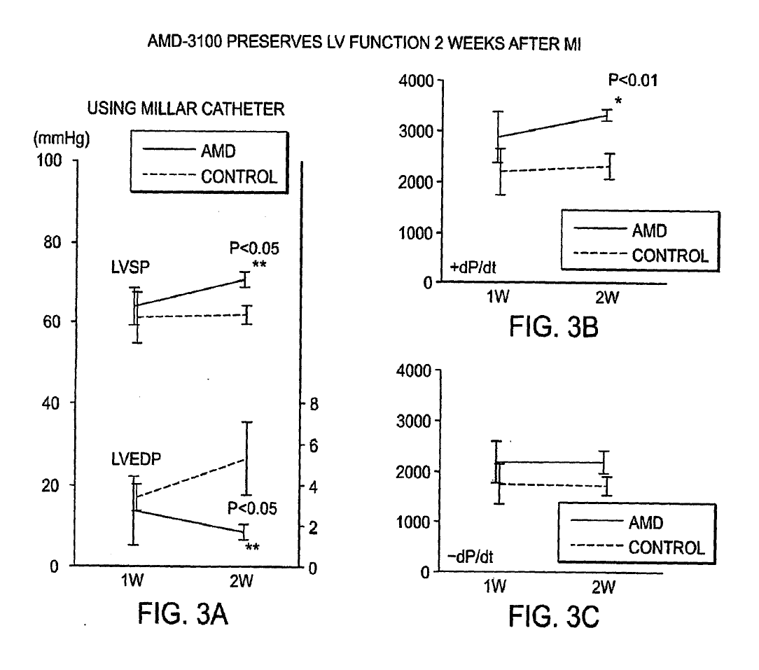

FIGS. 3A-3C show the results of hemodynamic measurements taken using a Millar catheter. Left ventricle (LV) systolic (71±2 mm Hg for AMD-3100 vs. 62±6 mm Hg for control) and end-diastolic (1.6±0.4 mm Hg for AMD-3100 vs. 5.3±1.8 mm Hg for control) pressures in the AMD-3100 group two weeks after AMI were improved, as compared to control (P<0.05). LV+dP/dt and −dP/dt, as an indicator of LV function, two weeks after AMI in the AMD-3100 group were also higher than control (+dP/dt: 3383±104 mm Hg/s for AMD-3100 vs 2354±255 mm Hg/s for control; −dP/dt: 2213±113 mm Hg/s for AMD-3100 vs. 1750±196 mm Hg/s for control, P<0.05).

Further data were obtained as follows. Hemodynamic parameters obtained from echocardiography and a Millar catheter from wild-type mice who received AMD3100 or saline are also summarized in Table 1. LVDd, LVDs, fractional shortening, and heart rate before MI and one week after MI were not significantly different between the groups.

TABLE 1
Hemodynamic Parameters in Mice Receiving AMD-3100 or Saline

| | | | After MI | | |
|---|---|---|---|---|---|
| Parameters | Group | Before MI | 1 week | 2 weeks | 4 weeks |
| LVDd, mm | AMD | 2.8 ± 0.1 | 3.5 ± 0.1 | 3.8 ± 0.1* | 4.0 ± 0.1* |
| | Control | 2.9 ± 0.1 | 3.7 ± 0.1 | 4.3 ± 0.1 | 4.5 ± 0.1 |
| LVDs, mm | AMD | 1.4 ± 0.1 | 2.2 ± 0.1 | 2.5 ± 0.1* | 2.9 ± 0.1* |
| | Control | 1.3 ± 0.1 | 2.7 ± 0.2 | 3.2 ± 0.1 | 3.5 ± 0.1 |
| FS, % | AMD | 52 ± 1 | 35 ± 2 | 34 ± 1* | 28 ± 1** |
| | Control | 53 ± 1 | 28 ± 3 | 25 ± 1 | 22 ± 1 |
| HR, bpm | AMD | 510 ± 26 | 494 ± 24 | 515 ± 16 | 473 ± 26 |
| | Control | 502 ± 14 | 519 ± 22 | 542 ± 15 | 523 ± 23 |
| LVSP, mmHg | AMD | | | 73 ± 3 | 71 ± 2** |
| | Control | | | 64 ± 4 | 62 ± 2 |
| LVEDP, mmHg | AMD | | | 1.5 ± 0.2** | 1.6 ± 0.4** |
| | Control | | | 3.4 ± 0.7 | 5.3 ± 1.8 |
| LV + dP/dt, mm Hg/s | AMD | | | 3475 ± 168** | 3383 ± 104* |
| | Control | | | 2550 ± 242 | 2353 ± 255 |
| LV − dP/dt, mm Hg/s | AMD | | | 2842 ± 200** | 2213 ± 112 |
| | Control | | | 1950 ± 242 | 1750 ± 196 |

Values are mean ± SE.
LVDd indicates left ventricular diastolic dimension;
LVDs, left ventricular systolic dimension;
FS, fractional shortening;
HR, heart rate;
LVSP, left ventricular systolic pressure;
LVEDP, left ventricular end-diastolic pressure;
LV, left ventricle.
*P < 0.01 vs Control,
**P < 0.05 vs Control.

Beginning at two weeks post-MI, echocardiography revealed decreased ventricular remodeling in the AMD3100-treated mice. LVDd (2 wks: 3.8±0.1 versus 4.3±0.1 mm, 4 wks: 4.0±0.1 versus 4.5±0.1 mm, P<0.01) and LVDs (3 wks: 2.5±0.2 versus 3.0±0.2 mm, 4 wks: 2.6±0.2 versus 3.2±0.2 mm, P<0.01). [Consider moving this to a table.] Fractional shortening at two and four weeks after MI was also significantly higher in the AMD group, compared with the control group (2 wks: 34±1 versus 25±1%; P<0.01, 4 wks: 28±1 versus 22±1%, P<0.05). There was no significant difference in heart rate between the two groups.

Hemodynamic measurements taken at two and four weeks after MI are shown in Table 1. Although there was no significant difference in left ventricular systolic pressure (LVSP) between the groups at two weeks after MI, left ventricular end-diastolic pressure (LVEDP) in the AMD group was significantly lower than in the control group (1.5±0.2 versus 3.4±0.7 mmHg, P<0.05). Moreover, four weeks after MI, LVEDP in the AMD group was also lower than in the control group (1.6±0.4 versus 5.3±1.8 mmHg, P<0.05). Nevertheless, LVSP in the AMD group was higher than in the control group (71±2 versus 62±2 mmHg, P<0.05). LV+dP/dt at two and four weeks after MI was significantly preserved in the AMD group compared with the control group (2 wks: 34751168 versus 2550±242 mmHg/s, P<0.05, 4 wks: 3383±104 versus 2353±255 mmHg/s, P<0.01). Such methods can be used for study of the compositions and methods of the instant invention. It is expected that use of the compositions and methods of the invention would also preserve left ventricle function two weeks after AMI.

### Example 6

### AMD-3100 Improves Survival Rate After AMI

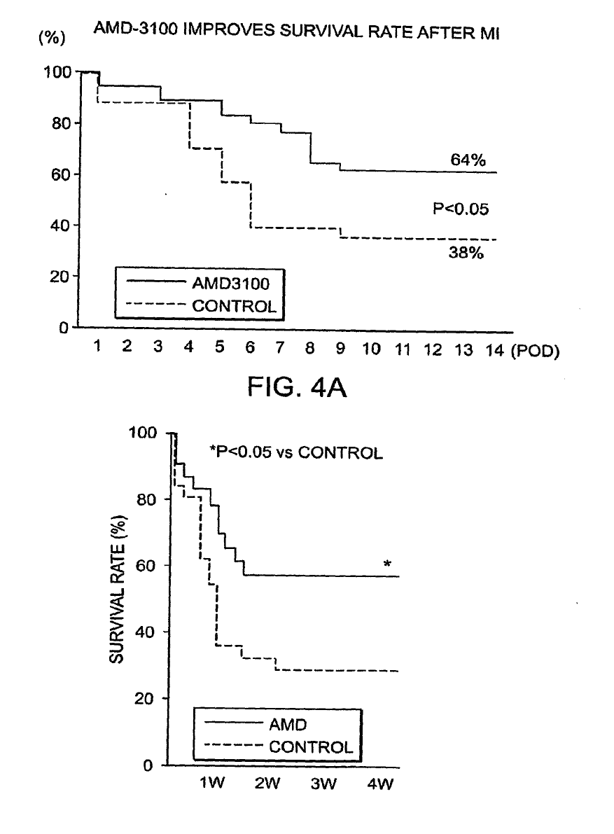

Survival rates of mice treated with AMD-3100 or saline (control) after AMI are shown in FIG. 4A. 64% of mice treated with AMD-3100 were still living 14 weeks after AMI, as compared to 38% of control mice (P<0.05).

In another group of mice, ten mice (42%) died in the AMD group, whereas 19 mice (70%) died in the control group. The survival rate four weeks after MI was higher in mice receiving AMD3100 than in mice receiving saline (P<0.05, FIG. 4B). Such methods can be used for study of the compositions and methods of the instant invention. It is expected that use of the compositions and methods of the invention would also improve survival rate after AMI.

### Example 7

### Increased Capillary Density in the Border Zone at One and Two Weeks After AMI

The explanted hearts of sacrificed mice were sliced in a bread-loaf fashion into transverse sections from apex to base (base-, mid-, and apex-portions) and fixed with 4% paraformaldehyde. Tissues were embedded in paraffin and sectioned for elastic tissue/trichrome staining and immunohistochemistry using the murine-specific EC marker Bandeiraea simplicifolia lectin I (Isolectin B4) (Vector laboratories).

Capillary density was assessed by histological examination of five randomly selected fields of tissue sections from segments of the left ventricular myocardium served by the occluded LAD. Capillaries were recognized as tubular structures positive for isolectin B4. Elastic tissue/trichrome-stained tissues from the mice four weeks after MI were used to measure the average ratio of the external circumference of fibrosis area to LV area.

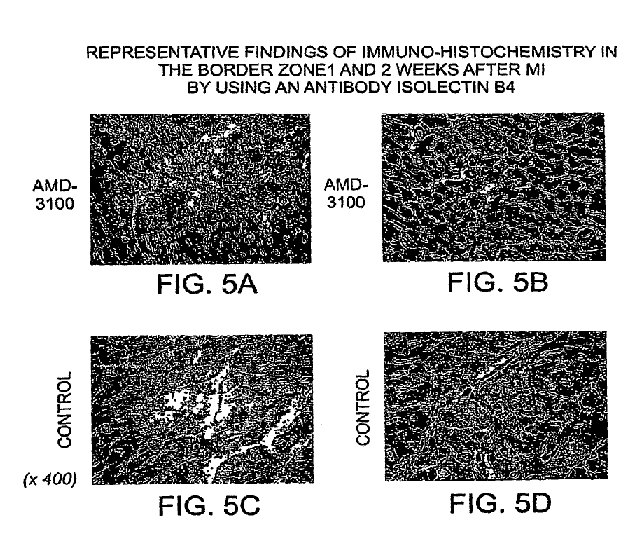

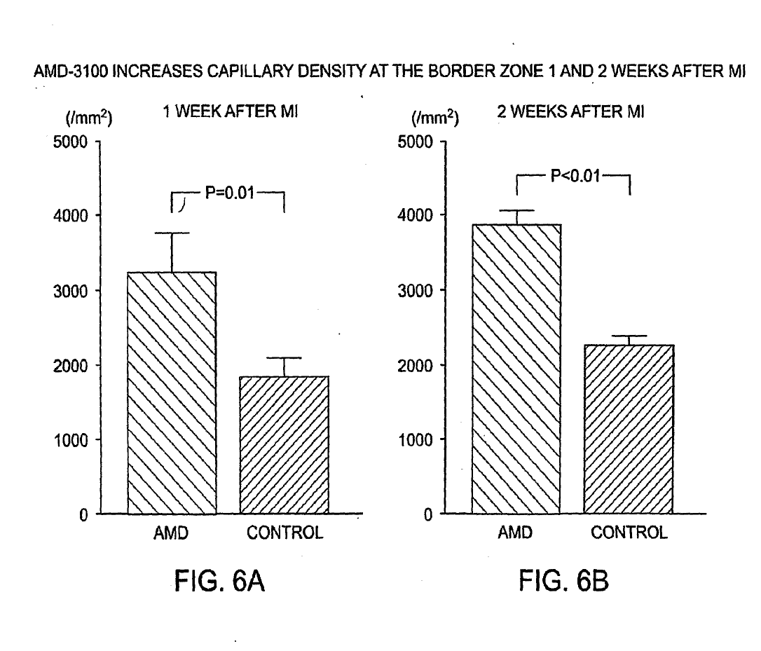

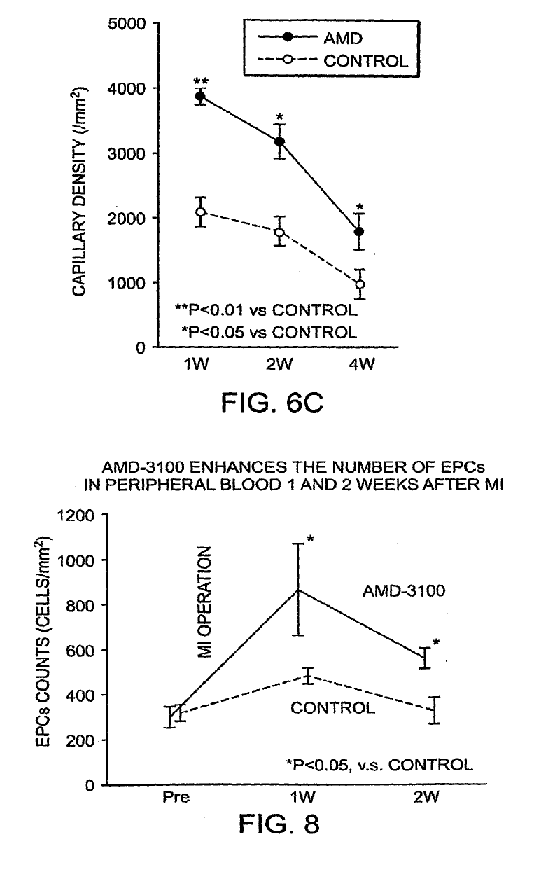

Immunohistochemistry of border zone sections using an antibody to isolectin B4 showed increased capillary density in the sections from AMD-3100-treated mice. Representative sections are shown in FIGS. 5A-5D. The increase (at two weeks, 4005±189/mm² for AMD-3100 vs. 2234±62/mm², P<0.01) is quantitated in FIGS. 6A-6C. Elastic trichrome-stained tissue in the control group four weeks after MI indicated marked dilatations of the LV cavity consistent with the echocardiographic measurements. Such methods can be used for study of the compositions and methods of the instant invention. It is expected that use of the compositions and methods of the invention would also increase capillary density after AMI.

### Example 8

### AMD-3100 Decreases Percent Fibrosis Area Two Weeks After AMI

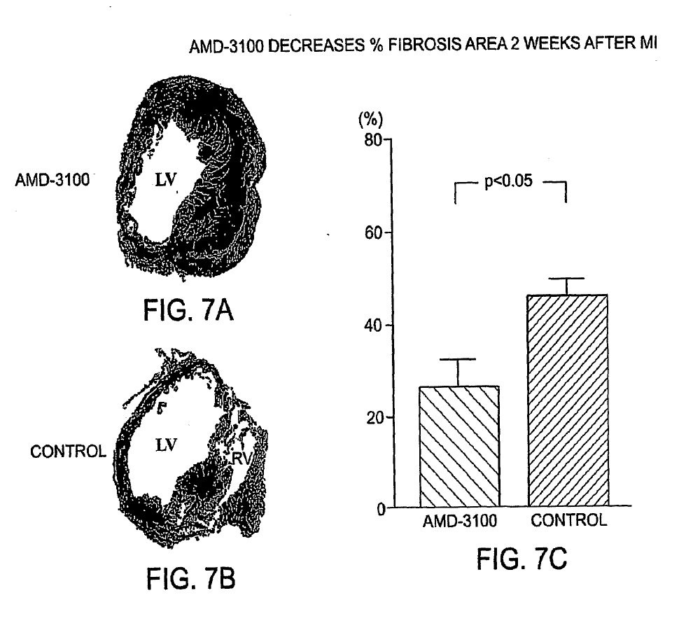

AMD-3100-treated and control mice were sacrificed two weeks after AMI. Sections of the left ventricles (FIGS. 7A-7B) showed a statistically significant decrease (P<0.05) in fibrosis area in the AMD-3100-treated mice (25±6% for AMD-3100 vs. 46±4% for control). This decrease is quantitated in FIG. 7C. Such methods can be used for study of the compositions and methods of the instant invention. It is expected that use of the compositions and methods of the invention would also decrease the percent of fibrosis area after AMI.

### Example 9

### AMD-3100 Enhances the Number of EPCs in Peripheral Blood One and Two Weeks After AMI

To evaluate the effect of AMD-3100 on circulating EPC kinetics, EPC counts (cells/m²) were taken from peripheral blood one and two weeks after AMI. At each time point after the induction of MI, mononuclear cells were isolated from peripheral blood and total BM cells that had been obtained by flushing the tibias and femurs. EPCs were counted by culturing as described in Example 1, and stained with both acLDL-DiI and BS-1 lectin. Double positive cells were counted in 15 fields under objective 20× magnification per sample in a blinded manner. AMD-3100-treated mice showed a statistically significant increase in the number of EPCs in the peripheral blood at both one (868±210/mm² for AMD-3100 vs. 485±33 mm² for control) and two weeks (556±47/mm² for AMD-3100 vs. 339±58/mm² for control) after AMI (P<0.05). The results are shown in FIG. 8. The number of cultured EPCs returned to basline at four weeks after AMI in both groups.

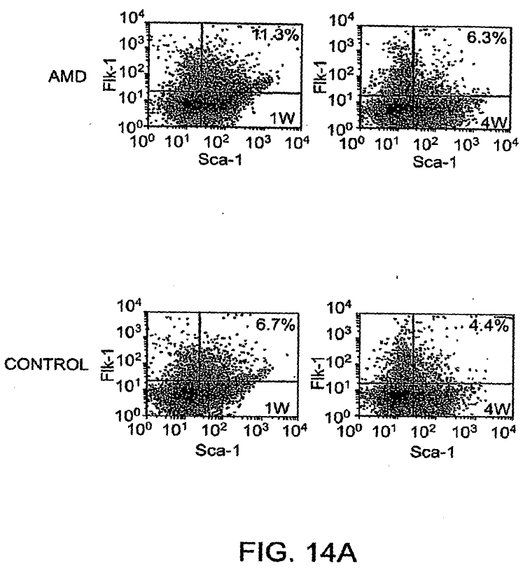

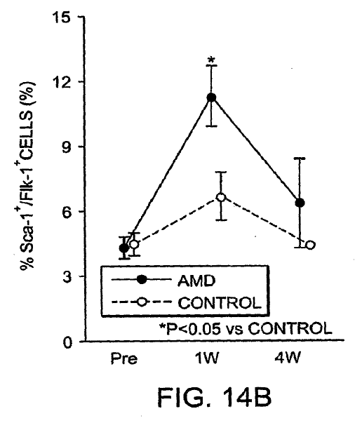

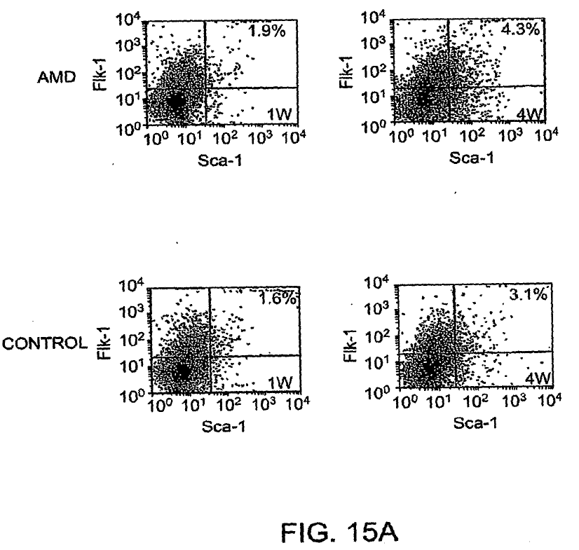

Fluorescence-activated cell sorting (FACS) was used to identify EPCs from mononuclear cells isolated both from peripheral blood and bone marrow. The viable mononuclear cell population was analyzed for the expression of Sca-1-FITC (Pharmingen) and Flk-1 (Santa Cruz) conjugated with the corresponding phycoerythrin-labeled secondary antibody (Sigma). Isotype-identical antibodies served as negative controls. Immunofluorescence-labeled cells were fixed with 2% paraformaldehyde and analyzed by quantitative flow cytometry using a FACStar flow cytometer (Becton Dickinson) and Cell Quest Software that counted 10,000 events per sample. Representative FACS analysis at one and four weeks after MI revealed a significant increase of EPCs in the AMD3100 group one week after MI, compared with the control group (11.3±1.4 versus 6.7±1.1%, P<0.05) (FIGS. 14A and 14C).

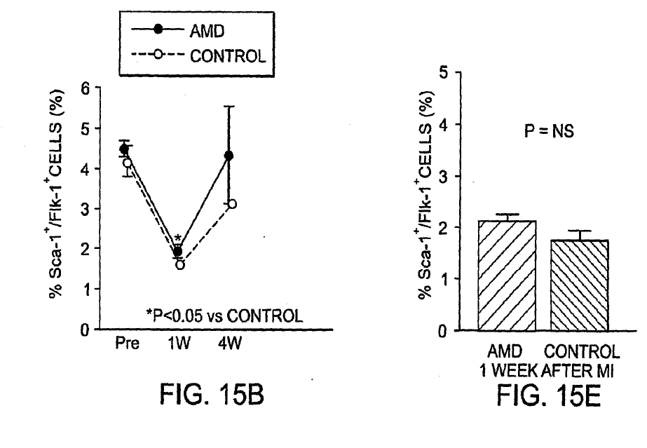

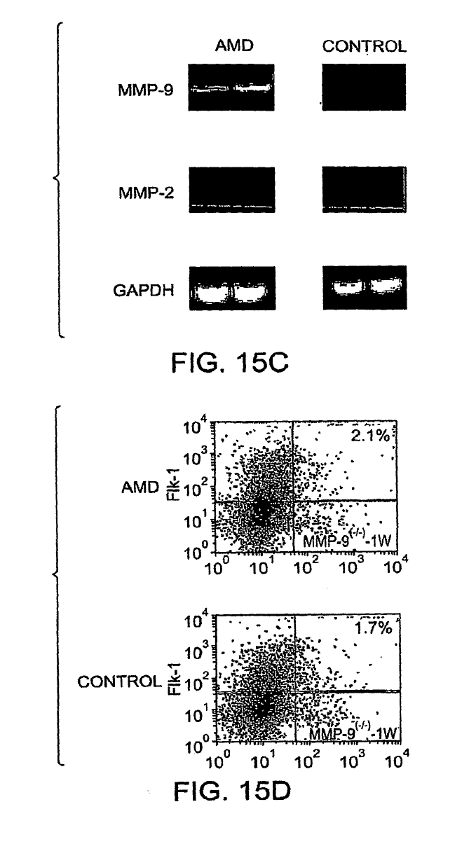

To examine the effect of AMD3100 on the kinetics of bone marrow EPCs after MI, BM mononuclear cells were isolated from total BM cells obtained by flushing the tibias and femurs. FACS analysis was used to identify Sca-1/Flk-1 positive cells as EPCs. Representative FACS analysis at one and four weeks after MI is shown in FIG. 15A. AMD3100 prevented a significant decrease of EPCs in the AMD group one week after MI, compared with the control group (1.9±0.1 versus 1.6±0.1%, P<0.05) (FIG. 15B)

To clarify the mechanism of BM-EPC kinetics induced by AMD3100, RT-PCR was performed for MMP-9 mRNA expression on BM-mononuclear cells one week after MI was performed. RNA was extracted from mononuclear cells isolated from bone marrow using TRIzol reagent (Invitrogen) according to the manufacture's instruction. Reverse transcriptase-polymerase chain reaction (RT-PCR) of the MMP-9 and GAPDH genes was performed using 1 pg of total RNA. PCR was performed for 25 cycles for both MMP-9 and GAPDH; each cycle maintained 94° C. for 30 seconds and 64° C. for three minutes. Amplification was carried out in 20-μl reaction mixtures containing 0.4 U Taq polymerase.

MMP-9 expression was disrupted by deleting of most of exon 2 of the MMP-9 gene in the 129/SvEv genetic background (Jackson), as described previously. [Vu, 1998 #2370] Male eight-week-old MMP^{-/-} mice randomly received either 125 μg AMD3100 or saline immediately after the induction of MI. Mononuclear cells from peripheral blood and total BM cells were isolated one week after MI and were examined by FACS analysis as described above.

RT-PCR detected enhanced expression of MMP-9 but not MMP-2 in BM-mononuclear cells (FIG. 15C). These data were strongly supported by FACS analysis for BM-mononuclear cells from M^{-/-} mice. Representative FACS analysis for quantification of Sca-1/Flk-1 positive cells is shown in FIG. 15D. There was no significant difference between mice receiving AMD3100 and those receiving saline (FIG. 15E). Such methods can be used for study of the compositions and methods of the instant invention. It is expected that use of the compositions and methods of the invention would also enhance the number of EPCs in peripheral blood after AMI.

### Example 10

### Experimental Design—Tie2-LacZ/BMT Model

This Example describes the experimental design of the Tie2-LacZ/BMT mouse model. These mice express LacZ in bone marrow-derived cells that express the endothelial cell specific marker Tie-2 (see Takahashi, T. et al. (1999) Nat. Genet. 5:434-438). FVB/N background mice (n=30) were irradiated to kill their endogenous bone marrow. They were then given a bone marrow transplant (BMT) of 2×10⁶ mononuclear cells (MNCs) derived from age-matched (4 weeks old) Tie2-LacZ mice (FVB/N-TgN[TIE2LacZ]182Sato, Jackson Lab). AMIs were induced in the mice (now called Tie2-LacZ/BMT mice) by LAD ligation four weeks after BMT, by which time the bone marrow of the recipient mice had been reconstituted. Right after AMI, the mice were each given a single subcutaneous injection of AMD-3100 (125 μg) or saline (control). Bone marrow cells were obtained using procedures described above. Bone marrow mononuclear cells were also isolated by density centrifugation. Tissue analysis was done at one or two weeks. Such methods can be used for study of the compositions and methods of the instant invention.

### Example 11

### AMD-3100 Augments Recruitment of Bone Marrow Derived EPCs One and Two Weeks After AMI

Tie-2/LZ/BMT mice were sacrificed at one and two weeks after MI. The explanted hearts were sliced in a bread-loaf fashion into transverse sections from apex to base (base-, mid-, and apex-portions). Mid-portions of the hearts were fixed in 4% paraformaldehyde for three hours at room temperature and incubated in X-gal solution overnight at 37° C. The target tissue samples were then placed in PBS [need to spell this out] and examined under a dissecting microscope for macroscopic identification of lacZ-expressing cells. Histological sections of the mid-portion heart tissue were counterstained with hematoxylin and eosin and assessed by light microscopy. The number of X-gal positive cells in six randomly chosen fields at 40× magnification were counted in a blinded manner.

Apex-sections from the Tie-2/LZ/BMT mice were embedded in OCT compound (Miles Scientific) and snap-frozen in liquid nitrogen for fluorescence microscopy immediately after sacrifice. Immunohistochemical staining was performed using antibodies against the rabbit anti-β-galactosidase IgG (CORTEX Biochem) and the murine-specific EC marker fluorescein griffonia simplicifolia lectin I (Vector Laboratories).

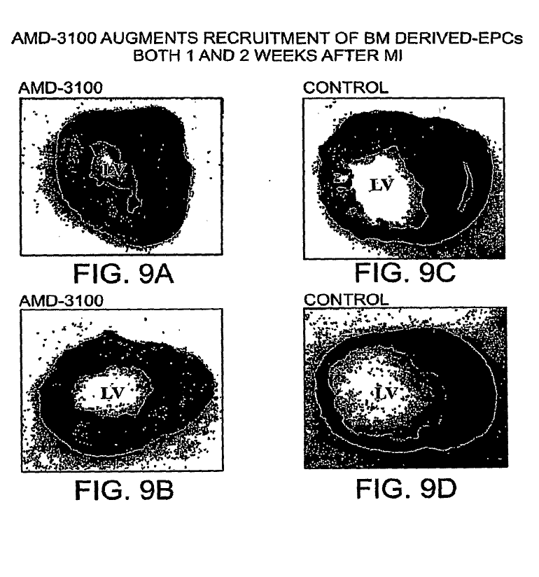

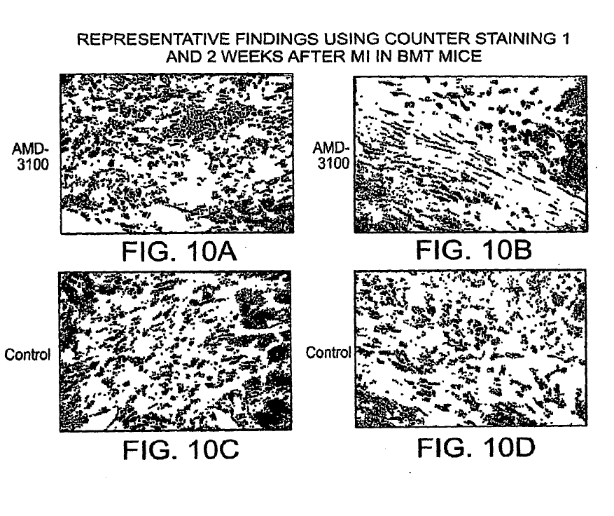

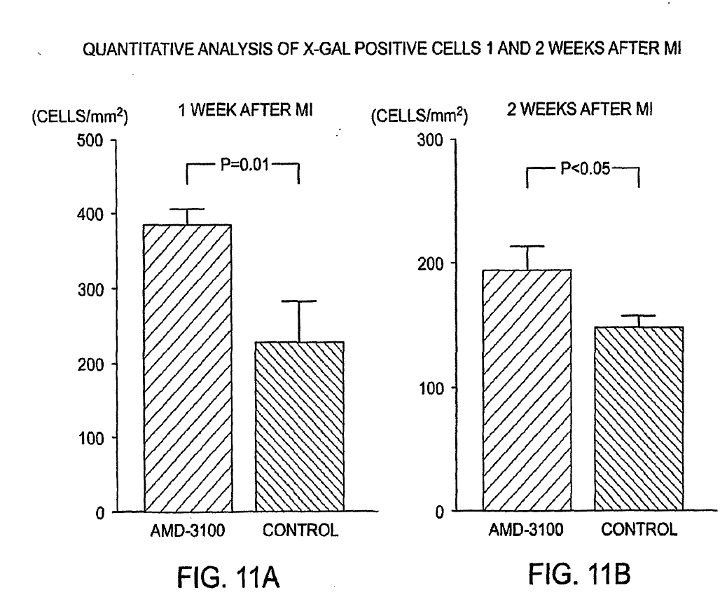

Left ventricle sections of AMD-3100-treated and control Tie2-LacZ/BMT mice were analyzed one and two weeks after AMI. Macroscopically, there was marked increase in X-gal staining of the left ventricle in the AMD-3100 group at one and two weeks, as compared to the controls (FIGS. 9A-9D). This indicates that there is an increase in the recruitment of bone marrow derived EPCs in the left ventricle after AMI in the treated group. Dilation of the left ventricle and the thickness of antero-lateral wall were similar between the two groups. Microscopic (400×) counterstaining showed similar results (FIGS. 10A-10D). A quantitative analysis of the number of X-gal positive cells is shown in FIGS. 11A-11B. The quantitative analysis of X-gal positive cells was evaluated by histological examination of six randomly selected fields in the infarct and border area. There is a statistically significant increase in the number of X-gal positive cells in the AMD-3100 treated cells, as compared to the controls, at both 1 and 2 weeks after AMI (1 week: 3776±113/mm² for AMD-3100 vs. 2231±323/mm² for control; 2 weeks: 1906±138/mm² for AMD-3100 vs. 1441±56/mm² for control; P<0.05). Such methods can be used for study of the compositions and methods of the instant invention. It is expected that use of the compositions and methods of the invention would also augment the recruitment of bone marrow derived EPCs after AMI.

### Example 12

### Fluorescence Microscopy on Myocardium One Week After AMI

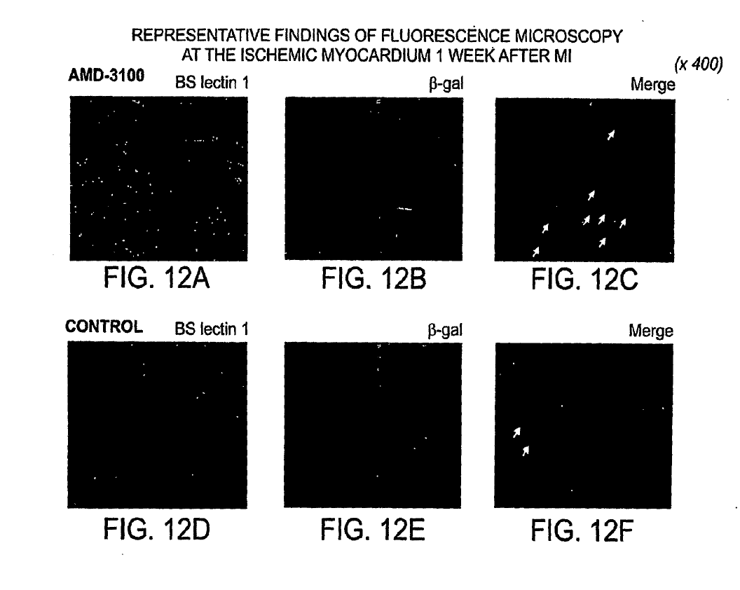

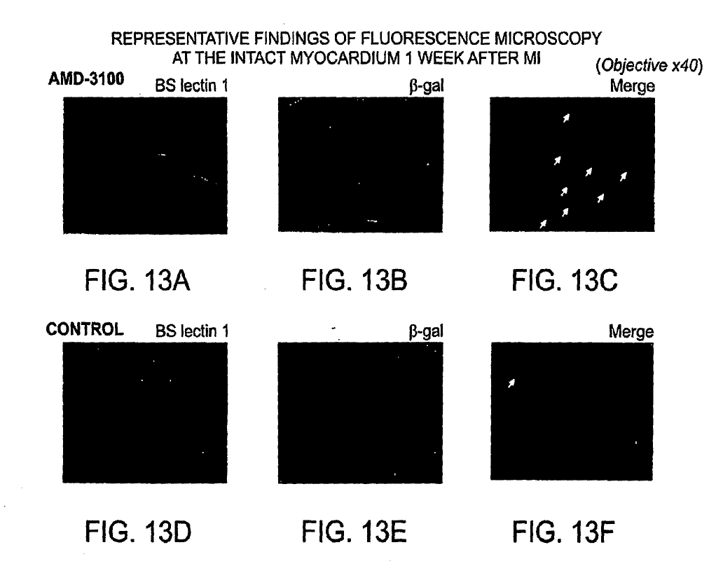

Sections of ischemic (FIGS. 12A-12F) and intact (FIGS. 13A-13F) myocardium were stained for both BS lectin 1 (green fluorescence) and β-galactosidase (red fluorescence). Merged fluorescence microscopy (400×) showed a marked increase in double-positive cells in tissue taken from AMD-3100-treated mice. In a representative section, the AMD-3100-treated mice had eight double-positive cells, as compared to one double-positive cell in the control section. In another representative section, the AMD-3100-treated mice had eight double-positive cells, as compared to two double-positive cells in the control section. Such methods can be used for study of the compositions and methods of the instant invention. It is expected that similar results would be seen using the compositions and methods of the instant invention.

### Example 13

### Statistical Analysis

All values were expressed as mean±the standard error of the mean. Statistical significance was evaluated using Wilcoxon Rank Sum Test for comparisons between mice receiving AMD3100 and those receiving saline. Overall survival of all the mice was assessed by Kaplan-Meier analysis. Survival in individual experimental groups was also analyzed by Fisher's extract probability test. A p value of less than 0.05 was considered statistically significant.

### Example 14

### Small Molecule CXCR4 antagonist and Sonic Hedgehog Gene Therapy Increase Arteriogenesis and Improve Cardiac Function in Chronic Myocardial Infarction

Pre-clinical and Pilot clinical trials have suggested that bone marrow-derived cell (BMC) may have therapeutic effects in acute myocardial infarction (MI). In contrast, the efficacy of BMC/EPC mobilization for repair of chronic MI is still controversial. A reason for the result is that angiogenic factors in myocardial tissue are decreased at the time remote from MI. There are reports that human Sonic hedgehog (Shh) gene increases angiogenic cytokine expression and enhances angiogenesis in myocardial ischemia model. An object in this example is to investigate the combination effect of CXCR4 antagonist AMD3100 injection as a bone BMC mobilization agent and Shh gene as an angiogenic gene for chronic MI.

Methods: Using wild-type rats, MI was induced by ligation of the left anterior descending artery. 4 weeks after MI induction, we randomized rats to 4 different treatments, AMD-3100 240 μg/kg SC twice/week (n7-9), Naked plasmid human Shh DNA (phShh) 300 ug/rat via intramyocardial injection (n=9), AMD and phShh combination (n=9) and saline s.c. injection as control (n=9). 4 weeks after treatment, echocardiography, hemodynamic measurement and histological examination were performed.

Results: Circulating EPC counts were five fold greater than baseline 2 hour after AMD treatment. Combination therapy attenuated the worsening of LV diastolic (1.0±0.05 cm vs. 1.2±0.06 cm p<0.05) and systolic (0.87±0.37 cm vs. 1.0±0.66 cm p<0.05) dimensions, % fractional shortening (+4.8% p<0.05) and LV dp/dt (+66% p<0.05). AMD treatment alone and combination therapy increased capillary density in the border zone between scar and normal tissue (+52% vs. control p<0.05). SDF-1 and Shh expression are increased greater in myocardium 4 week after injection in combination therapy group compare with control.

Accordingly, even at a time remote from myocardial infarction, combination of bone marrow cell mobilization therapy and angiogenic gene therapy using CXCR4 antagonist and SHh gene has the potential to improve cardiac dysfunction via mobilization of EPC and activation of angiogenic cytokine in myocardial tissue.

Those skilled in the art will recognize, or be able to ascertain using no more than routine experimentation, many equivalents to the specific embodiments of the invention described herein. Such equivalents are intended to be encompassed by the following claims.

What is claimed:

1. A method of preventing, treating, and/or alleviating symptoms associated with tissue ischemia, the method comprising administering to a subject in need thereof a therapeutically effective amount of (a) at least one CXCR4 antagonist and (b) at least one nucleic acid encoding at least one morphogen or an effective fragment of a morphogen, to prevent, treat, or alleviate the symptoms associated with the tissue ischemia in the subject.

2-14. (canceled)

15. The method of claim 1, wherein the CXCR4 antagonist has the formula:
Z-linker-Z′
or a pharmaceutically acceptable salt thereof
wherein Z is a cyclic polyamine containing 9-32 ring members of which 3-8 are nitrogen atoms, said nitrogen atoms separated from each other by at least 2 carbon atoms, and wherein said heterocycle may optionally contain additional heteroatoms besides nitrogen and/or may be fused to an additional ring system;
wherein Z′ may be embodied in a form as defined by Z above, or alternatively may be of the formula
—N(R)—(CR₂)ₙ—X (1)
wherein each R is independently H or straight, branched or cyclic alkyl (1-6C), n is 1 or 2, and X is an aromatic ring, including heteroaromatic rings, or is a mercaptan; and
wherein “linker” represents a bond, alkylene (1-6C) or may comprise aryl, fused aryl, oxygen atoms contained in an alkylene chain, or may contain keto groups or nitrogen or sulfur atoms.

16. The method of claim 15, wherein Z and Z′ are both cyclic polyamines.

17. The method of claim 15, wherein Z and Z′ are identical.

18. The method of claim 15, wherein Z contains 12-24 members and contains 4 nitrogen atoms.

19. The method of claim 15, wherein Z and Z′ are both 1,4,8,11-tetraazocyclotetradecane.

20. The method of claim 15, wherein the linker comprises an aromatic ring bracketed by two methylene moieties.

21. The method of claim 15, wherein the linker is 1,4-phenylene-bis-methylene.

22. The method claim 15, wherein the CXCR4 antagonist is 1,1′-1,4-phenylene-bis-(methylene)-bis-1,4,8,11-tetraazacyclotetradecane (AMD-3100).

23. The method of claim 15, wherein the CXCR4 antagonist is selected from the group consisting of:
N-[1,4,8,11-tetraazacyclotetradecanyl-1,4-phenylenebis(methylene)]-2-aminomethyl)pyridine;
7,7′-[1,4-phenylenebis(methylene)]bis-4,7,10,17-tetraazabicyclo-[13.3.1]heptadeca-1(17),13,15-triene;
7,7′-[1,4-phenylenebis(methylene)]bis-3,7,11,17-tetraazabicyclo[13.3.1]heptadeca-[(17),13,15-triene;
1,1′-[1,3-phenylenebis(methylene)]-bis-1,4,8,11-tetra-azacyclotetradecane;
1,1′-t-1,4-phenylenebis(methylene)]-bis-1,4,8,11-tetra-azacyclotetradecane;
1,1′-[1,4-phenylene-bis-(methylene)]-bis-1,4,7,10-tetraazacyclotetradecane;
1,1′-[1,3-phenylene-bis-(methylene)]-bis-1,4,7,10-tetraazacyclotetradecane; 11,11′-(1,2-propanediyl)bis-1,4,8,1-tetraazacyclotetradecane;
N-[4-(1,4,7-triazacyclotetra-decane)-1,4-phenylenebis(methylene)]-2-(aminomethyl)pyridine;
N-[7-(4,7,10-triazabicyclo[13.3.1]heptadeca-1(17),13,15-triene)-1,4-phenylenebis(methylene)]-2-(aminomethyl)pyridine;
N-[7-(4,7,10,17-tetraazabicyclo[13.3.1]heptadeca-1(17), 13,15-triene)-1,4-phenylenebis(methylene)]-2-(aminomethyl)pyridine; and
N-[4-[4,7,10,17-tetraazabicyclo[13.3.1]heptadeca-1(17), 13,15-triene]-1,4-phenylenebis(methylene)]-2-(aminomethyl)pyridine.

24-26. (canceled)

27. The method of claim 1, wherein the method further comprises the step of mobilizing in the subject an endothelial progenitor cells (EPCs) and/or a bone marrow-derived stem cells (BMSCs).

28-35. (canceled)

36. A method for inducing new blood vessel growth in myocardial tissue of a mammal in need of such treatment comprising administering a therapeutically effective amount of (a) at least one CXCR4 antagonist; and (b) at least one nucleic acid encoding at least one morphogen or an effective fragment of a morphogen.

37. The method of claim 36, wherein the method further comprises selecting a patient having, suspected of having, and/or prone to ischemia and administering the nucleic to or near a heart or limb blood vessel in need of treatment.

38. (canceled)

39. The method of claim 36, wherein the method further comprises administering the nucleic with a stent, catheter, implementation for performing balloon angioplasty; or related device.

40. The method of claim 36, wherein the myocardial tissue has been impacted by one of ischemia, infarction or dysfunction.

41-44. (canceled)

45. A pharmaceutical product for preventing, alleviating, or treating an ischemic disorder in a mammal, the product comprising (a) at least one CXCR4 antagonist; and (b) at least one nucleic acid encoding at least one morphogen, or at least one nucleic acid encoding at least an effective fragment of a morphogen, wherein each the antagonist and the nucleic acid are formulated to be physiologically acceptable to a mammal, the product further comprising means for administering the product to the mammal.

46-47. (canceled)

48. A kit comprising the pharmaceutical product of claim 45, the kit further comprising a pharmacologically acceptable carrier solution, and means for delivering at least the morphogen to the mammal and directions for using the kit.

49-50. (canceled)

51. A method for increasing production of EPCs and/or BMSCs in a subject, the method comprising administering a therapeutically effective amount of at least one of a CXCR4 antagonist and a nucleic acid encoding at least one of an N-terminal portion of human hedgehog (SHh) protein, human desert hedgehog (DHh) protein or human Indian Hedgehog (IHh) protein; the method further comprising increasing presence of the EPCs and/or the BMSCs in the subject.

52. (canceled)

53. A use of a composition for preparation of a medicament for preventing, treating, and/or alleviating symptoms associated with tissue ischemia, the composition comprising a therapeutically effective amount of (a) at least one CXCR4 antagonist; and (b) at least one nucleic acid encoding at least one morphogen or an effective fragment of a morphogen.

54. The use of claim 53, wherein the composition is formulated for co-administration of the CXCR4 antagonist and the nucleic acid.

55. (canceled)
